(19) United States
(12) Patent Application Publication
(10) Pub. No.: US 2022/0192993 A1
(43) Pub. Date: Jun. 23, 2022

(54) DOSAGE FORMS AND METHODS OF PREPARATION AND USE THEREOF

(71) Applicant: Willow Bark Brands, Inc., New York, NY (US)

(72) Inventor: Eric R. First, Morristown, NJ (US)

(21) Appl. No.: 17/440,167

(22) PCT Filed: Apr. 3, 2020
(86) PCT No.: PCT/US2020/026512

§ 371 Date: Sep. 16, 2021

Related U.S. Application Data
(60) Provisional application No. 62/828,783, filed on Apr. 3, 2019.

(51) Int. Cl. A61K 9/28; A61K 31/015; A61K 31/198; A61K 31/23; A61K 31/352; A61K 45/06; A61K 36/575; A61K 36/756; A61K 36/81

(52) U.S. Cl. CPC A61K 9/282; A61K 31/015; A61K 31/198; A61K 31/23; A61K 36/81; A61K 45/06; A61K 36/575; A61K 36/756; A61K 31/352

(57) ABSTRACT

Disclosed are dosage forms and kits that support the human body during a stress response. The dosages forms can include at least one sensory disrupter, at least one stress-buster and at least one calming reliever. In embodiments, the dosage form can include at least one nervous system component, at least one immune system component, and at least one endocrine system component. Also disclosed are kits containing the dosage forms and methods of preparation and use of such dosage forms.

### FIELD

Disclosed herein are dosage forms, dietary supplements and methods of preparation and use thereof. The formulations and methods of use help regulate a human body's response to stress to help reduce associated stress and anxiety.

### BACKGROUND

There are many treatments for stress and anxiety including the use of benzodiazepines, anti-depressants and various herbs. However, prolonged use of benzodiazepines can lead to dependence, as well as unwanted effects such as drowsiness and foggy thinking. Anti-depressants may be helpful to some people to help stress and anxiety, but they are specifically intended for depression. Some anti-depressants, such as Selective Serotonin Reuptake Inhibitors (SSRIs) can have adverse effects such as agitation, weight gain and other side-effects. Various herbal supplements have been reported to modulate stress and anxiety; however, the intended targets typically include one or more targets within the central nervous system (CNS).

Experts have known about the effects of stress on the body and its impact on the immune system for some time, but recently researchers have been looking more deeply into what happens in the brain to cause stress, and the brain's function during a period of stress. Inflammation can damage the brain, just as it can damage other organs and systems, and studies have previously linked chronic stress to a higher risk of dementia and Alzheimer' s in addition to anxiety and other mood disorders.

Stress can also impact how the brain functions. When an individual is in “fight-or-flight mode,” more energy, nutrients, and resources are funneled to parts of the brain associated with survival and dealing with threats. Meanwhile, other areas, including those responsible for creating memories and high-level cognitive processing, receive the bare minimum. If stuck in that survival mode long-term, the brain may actually rewire. A recent study in the journal Neurology reported on how damaging to the brain chronic stress can be. It examined cortisol levels, brain size and structure, plus memory and cognitive functioning in more than 2,000 people in their 40s and 50s. The researchers found that those with higher levels of cortisol in their systems had smaller brains, and they also scored worse on memory and cognitive tests—even though none of the participants exhibited noticeable symptoms of cognitive decline or memory problems. More specifically, changes and damage were seen in parts of the brain that move information between the right and left hemispheres as well as areas associated with thought, speech, emotion, and muscle function.

These findings indicate that the effects of a high-stress life may start causing problems in the brain much earlier in life than previously thought, and in ways that had not yet been known. While more research is needed to determine what exactly this may mean for people as they age, one thing is clear: new methods to modulate the stress response are needed.

### BRIEF DESCRIPTION OF DRAWINGS

The figures are not necessarily to scale, emphasis instead generally being placed upon illustrative principles. The figures are to be considered illustrative in all aspects and are not intended to limit the disclosure, the scope of which is defined only by the claims.

FIG. 1 illustratively depicts a flowchart of a typical stress response in a human being.

FIG. 2A illustratively depicts a dosage form according to embodiments herein.

FIG. 2B illustratively depicts a kit according to embodiments herein.

FIG. 3A illustratively depicts a dosage form according to embodiments herein.

FIG. 3B illustratively depicts kit according to embodiments herein.

FIG. 4 illustratively depicts a flowchart showing the mediation of stress in the body upon administration of a dosage form and/or kit according to embodiments herein.

FIG. 5 illustratively depicts a flowchart showing the synergistic combination of effects from dosage forms and kits according to embodiments herein.

FIG. 6 illustratively depicts a flowchart showing a 14 day administration cycle of dosage forms and kits according to embodiments herein.

### BRIEF SUMMARY

According to embodiments, disclosed herein are dosage forms, comprising a sensory disrupter, for example, at least one of a terpene, linalool, limonene and caryophyllene; a stress-buster, for example, at least one of arginine, lysine, glycine, gamma amino butyric acid (GABA), magnesium, citi-choline, inositol and apigenin; and a calming reliever, for example, at least one of cannabidiol, lemon balm, passionflower and ashwagandha. In embodiments, the plurality of units comprise a first plurality of units and a second plurality of units. In embodiments, the plurality of units provide a controlled release of components within the plurality of units. According to embodiments, the plurality of units comprise at least one of particles, granules, beads, microspheres, micro-tablets and extrudates. In embodiments, the first plurality of units is a different color from the second plurality of units, optionally, the color is derived from at least one of beets and spirulina. In embodiments, the plurality of units are contained in a pharmaceutically acceptable capsule, optionally, wherein the capsule comprises a mixture of natural oils and components, and optionally, wherein the capsule provides an immediate release of the natural oils and components. In various embodiments, the capsule is clear or translucent. The capsule may contain a coating thereon, optionally wherein the coating comprises at least one aromatic oil. According to various embodiments, the dosage form provides an immediate release of at least one of the sensory disrupter, the stress-buster and the calming reliever. In embodiments, the dosage form provides a controlled release of at least one of the sensory disrupter, the stress-buster and the calming reliever. In certain embodiments, the dosage form provides an immediate release of at least one of the sensory disrupter, the stress-buster and the calming reliever and a controlled release of at least one of the sensory disrupter, the stress-buster and the calming reliever. In yet further embodiments, the dosage form is free of at least one of melatonin, diphenhydramine, caffeine, cannabinoids, cypress oil, eucalyptus oil, tea tree oil, rose oil and sandalwood oil.

According to various embodiments, disclosed herein are dosage forms, comprising a nervous system component, for example, at least one of gamma amino butyric acid, valerian root, 5-hydroxytryptophan, L-theanine, passionflower, and lemon balm; an immune system component, for example, at least one of Ahi flower oil, medium chain triglyceride (MCT) oil, Vitamin D3, an aromatic oil; and an endocrine system component, for example, at least one of magnolia extract, phaelladendron extract, and ashwagandha. In embodiments, the dosage form comprises a plurality of units, optionally wherein the plurality of units comprises a first plurality of units and a second plurality of units. In embodiments, the plurality of units comprise at least one of particles, granules, beads, microspheres, micro-tablets and extrudates. In embodiments, the first plurality of units is a different color from the second plurality of units. In embodiments, the plurality of units are contained in a pharmaceutically acceptable capsule, optionally wherein the capsule is clear or translucent. In embodiments, the capsule contains a coating, optionally comprising at least one aromatic oil. In embodiments, the dosage form provides an immediate release of at least one of the nervous system component, the immune system component and the endocrine system component. In embodiments, the dosage form provides a controlled release of at least one of the nervous system component, the immune system component and the endocrine system component. In embodiments, the dosage form provides an immediate release of at least one of the sensory disrupter, the stress-buster and the calming reliever and a controlled release of at least one of the sensory disrupter, the stress-buster and the calming reliever.

According to various embodiments, dosage forms as described herein are formulated for any suitable route of administration. In embodiments, the dosage forms comprise a route of administration selected from oral, parenteral, subcutaneous, intravenous, intramuscular, intraperitoneal, transdermal, transmucosal (e.g., via the transurethral or rectal routes), sublingual, buccal, gingival, rectal, subcutaneous, transpulmonary, topical, nasal, inhalation and combinations thereof. In embodiments, dosage forms as described herein include at least one additional active ingredient. The at least one additional active ingredient can include at least one of an anti-anxiety drug, an antidepressant, a cannabinoid, dronabinol, a prodrug thereof, a pharmaceutically acceptable salt thereof and combinations thereof.

According to embodiments, disclosed herein is a dosage form, comprising the following ingredients: vitamin D3; ahi flower; MCT oil, L-Theanine; 5-hydroxytryptophan; valerian root; a mixture of Phellodendron amurense and Magnolia officinalis; gamma amino butyric acid; ashwagandha; beta caryophyllene; citrus oil; and mint oil. Optionally, the dosage form is a pharmaceutically acceptable capsule comprising a combination of natural oils and ingredients, optionally, the capsule provides an immediate release of the natural oils and ingredients, and optionally, the capsule is translucent or clear. In embodiments, optionally the ingredients are in the form of a plurality of units, optionally, wherein a first plurality of the plurality of units is a different color from a second plurality of the plurality of units, and optionally, wherein the plurality of units provide a controlled release of the ingredients. In embodiments, the dosage form comprises at least one additional active ingredient.

According to embodiments, disclosed herein is a dosage form, comprising vitamin D3; ahi flower; MCT oil; passionflower; lemon balm; chamomile flower; valerian root; a mixture of Phellodendron amurense and Magnolia officinalis; gamma amino butyric acid; ashwagandha; beta caryophyllene; lavender oil; and mint oil. Optionally, the dosage form is a pharmaceutically acceptable capsule comprising a combination of natural oils and ingredients, optionally, the capsule provides an immediate release of the natural oils and ingredients, and optionally, the capsule is translucent or clear. In embodiments, the ingredients are in the form of a plurality of units, optionally, wherein a first plurality of the plurality of units is a different color from a second plurality of the plurality of units, and optionally, wherein the plurality of units provide a controlled release of the ingredients. In embodiments, the dosage form comprises at least one additional active ingredient.

According to various embodiments, disclosed herein are kits, comprising a container; and at least one dosage form as described herein, removably positioned within the container. In embodiments, the dosage form comprises at least one sensory disrupter; at least one stress-buster; and at least one calming reliever. In embodiments, the dosage form comprises at least one nervous system component; at least one immune system component; and at least one endocrine system component. In embodiments, the container comprises at least one of a textured material, a soft material, a wood material, a bamboo material and a calming color that, upon touch, sight or both, provides a sensory disruption to a human. In embodiments, the kit comprises an aromatic oil within the container, the aromatic oil can be within a canister, coated on an inner surface of the container, coated on an outer surface of the container, coated on an outer surface of the dosage form, contained within the dosage form, or a combination thereof. In embodiments, the kit further includes external packaging, wherein the container is removably positioned within the external packaging. The external packaging can include at least one of a textured material, a smooth material, a soft material, a wood material, a bamboo material and a calming color that, upon touch, sight or both, provides a sensory disruption to a human. In embodiments, the kit can further include an aromatic oil within the external packaging. The aromatic oil can be within a canister, coated on an inner surface of the container, coated on an outer surface of the container, or a combination thereof. In embodiments, the kit includes instructions for using the kit to reduce at least one of stress and anxiety. The instructions can direct a human to open the container and hold under nose, exhale then breathe in through the nose drawing breath all the way into belly; pause for about 1 second to about 10 seconds, then exhale from chest, imagine belly button touching spine, optionally, repeat breathing and exhaling; and insert one dosage form into mouth, taste the dosage form while swallowing.

According to various embodiments, disclosed are methods of using dosage forms and kits as described herein. In embodiments, disclosed are methods of supporting a human body's response to stress, comprising: administering to a human a dosage form according to embodiments here. The dosage form can be administered by the human as needed when the human anticipates stress, anxiety or both. The dosage form can be administered by the human in the afternoon or evening. In embodiments, the human is administered up to 10 servings (or up to 4 servings) daily of the dosage form.

Further disclosed are methods of supporting a human's response to anxiety disorder, comprising: administering to a human a dosage form according to embodiments herein. The dosage form can be administered by the human as needed when the human anticipates stress, anxiety or both. The dosage form can be administered by the human in the afternoon or evening. The human can be administered up to 10 servings daily, or up to four servings daily, of the dosage form.

According to various embodiments, disclosed herein are methods of supporting a human's response to stress or anxiety, comprising inducing a sensory stimulation invoking a sensory nervous system response, the sensory stimulation causing an acute reduction in stress in a human; delivering ingredients to target key receptors or other targets within the central nervous system, the ingredients indirectly driving an autonomic nervous system response causing a longer term reduction in stress of the human, wherein the inducing sensory stimulation and delivering ingredients causes a dual stimulation; and providing, based on the dual stimulation induced by the sensory stimulation and delivered ingredients, a synergistic relaxation response modulated by the autonomic nervous system. In embodiments, the sensory stimulation provides a sensory distraction.

### Definitions

In describing the present disclosure, the following terms are to be used as indicated below. As used herein, the singular forms “a,” “an,” and “the” include plural references unless the context clearly indicates otherwise. Thus, for example, reference to “an active agent” includes a single active agent as well as a mixture of two or more different active agents, and reference to “a resin” includes a single resin as well as a mixture of two or more different resins, and the like.

The use of the terms “include,” “includes,” “including,” “have,” “has,” or “having” should be generally understood as open-ended and non-limiting unless specifically stated otherwise.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything higher than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

As used herein, the terms “component,” “ingredient,” “active agent,” “active ingredient,” “pharmaceutical agent,” and “drug” refer to any material that is intended to produce a therapeutic, prophylactic, and/or other intended effect to a human in need thereof, whether or not approved by a government agency for that purpose. These terms with respect to specific agents include all active forms of the agent, including the free base form of the agent, and all pharmaceutically acceptable salts, complexes, stereoisomers, crystalline forms, co-crystals, ether, esters, hydrates, solvates, prodrugs and mixtures thereof, where the form is active.

For purposes of this disclosure, “pharmaceutically acceptable salts” include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate and the like; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt and the like.

The terms “controlled-release,” “extended-release” or “sustained release” are interchangeable and are defined for purposes of the present disclosure as the release of one or more component (e.g., a sensory disrupter, a stress buster, a calming reliever, etc.) at such a rate that blood (e.g., plasma) concentrations are maintained within the therapeutic range, but below toxic concentrations, over a period of time of at least about 1 hour or longer, at least about 2 hours or longer, at least about 3 hours or longer, at least about 4 hours or longer, at least about 5 hours or longer, or at least about 6 hours or longer, or at least about 10 hours or longer, or at least about 12 hours or longer or at least about 24 hours or longer.

The term “immediate release” is defined for the purposes of the present disclosure as the release of one or more component (e.g., a sensory disrupter, a stress buster, a calming reliever, etc.) into the air, mouth or in the gastrointestinal contents with no delay or prolongation of dissolution or absorption, smell or taste of the one or more component.

### DETAILED DESCRIPTION

Embodiments of the present disclosure provide formulations and methods of preparation and use thereof that help regulate a human body's response to stress and anxiety. In embodiments, a dosage form, for example, a dietary supplement, includes a sensory disrupter, a stress-buster and a calming reliever. The sensory disrupter can include at least one of a terpene, linalool, limonene and caryophyllene. The stress-buster can include at least one of arginine, lysine, glycine, gamma amino butyric acid (GABA), magnesium, citi-choline, inositol and apigenin. The calming reliever can include at least one of a cannabidiol, lemon balm, passionflower and ashwagandha. In embodiments, the dosage form, for example, a dietary supplement, can include a nervous system component, an immune system component and an endocrine system component. The nervous system component can include at least one of gamma amino butyric acid, valerian root, 5-hydroxytryptophan, L-theanine, passionflower, and lemon balm. The immune system component can include at least one of Ahi flower oil, MCT oil, Vitamin D3 and an aromatic oil. The endocrine system component can include at least one of magnolia extract, phaelladendron extract and ashwagandha.

According to further embodiments, disclosed herein are methods of use that employ a multi-targeted approach that invokes the afferent sensory branch within the peripheral nervous system division and the central nervous system (CNS) to treat stress acutely and mid-long term. In embodiments, the methods of use include administering the dosage form or a kit including the dosage form to provide a “sensory distraction,” the acute response, which causes a physiologic response that temporarily reduces stress. In addition to containing a sensory distraction component, the dosage form can include one or more stress relieving ingredients that target key receptors or other targets within the CNS and indirectly affect the autonomic nervous system (ANS) to reduce stress over the mid- to longer-term, i.e., the sustained response. Notably, within the ANS division there are two branches: the sympathetic (i.e., activates stress response) and the parasympathetic (i.e., counteracts stress response, e.g., the relaxation response). This dual approach of invoking changes of input to the parasympathetic nervous system (PNS) and CNS, surprisingly provides a synergistic relaxation response modulated by the ANS (i.e., the parasympathetic branch), thus reducing stress in the short term (immediate), as well as a controlled (mid-long term) response.

Although the disclosure relates to different aspects and embodiments, it is understood that the different aspects and embodiments disclosed herein can be integrated, combined, or used together as a combination system, or in part, as separate components, devices, and systems, as appropriate. Thus, each embodiment disclosed herein can be incorporated in each of the aspects to varying degrees as appropriate for a given implementation.

### Stress Response

A stress response provides a burst of energy to fight off attackers or run away effectively. This helped human ancestors, who faced numerous physical threats, to stay safe. In those times, the main threats were physical in nature and short-lived, usually predators indicating an extreme physical threat that then vanish.

However, current threats tend not to be physical and instead associated with the modern way of life—a challenge to status, a demand for performance, or any situation where the demand may exceed a human's ability to cope or require the human to work on coping. In addition to providing a set of changes that may not match current needs (e.g., it might be more effective to have a burst of mental clarity or wisdom than a burst of physical strength when facing a psychosocial stressor), the stress response can actually cause harm if it leads to a state of chronic stress—that is, if a stress response is triggered and sustained, then the body does not go back to its normal state via the relaxation response.

The strength of the stress response is related to the level of perceived threat rather than actual, physical threat. As such, two people can experience the same situation and have very different stress reactions to the same trigger—some people perceive a threat where others do not. Knowing this, people may reduce the strength of their stress response by reminding themselves that this threat may not be as immediate as perceived. This is difficult to do, however, particularly for those who do not realize such a possibility exists.

A person may experience a greater level of stress when ignored or in a tense social situation, versus when they are driving a car in congested traffic, where the chances of being physically hurt are greater. Some people may experience greater levels of stress when speaking in public with no actual threat of physical danger (and relatively little social danger), but they nonetheless feel threatened and find their hands sweating and shaking, and their feet cold as the epinepherine/norepinephrine is released and redirection of blood flow away from extremities yields its effects. This also comes into play when negative experiences in childhood become stress triggers later in life causing a feeling that the person may be similarly hurt, but actually is not in danger.

### Stress Pathways

There are a number of systems that impact the degree of stress and anxiety a person may experience. The main “stress pathways” include:

The hypothalamic-pituitary adrenal axis (HPA-axis). When feeling stressed, the body goes into a “fight or flight” response and secretes increased amounts of stress hormones including cortisol and epinepherine/norepinephrine. These hormones are released through the HPA axis (a central hormone pathway), which includes the hypothalamus, the pituitary gland, and the adrenal glands. When stress levels are high, they lead to an imbalance in the HPA axis.

The Catecholamine Pathways: Three commonly known catecholamines are epinephrine (adrenaline), norepinephrine, and dopamine. Catecholamines can serve as neurotransmitters, transferring signals from neuron to neuron, as well as hormones, which regulate physiological functions such as heartbeat and breathing rate.

The Corticosteroid Pathway The hypothalamus signals the pituitary gland to secrete adrenocorticotropic hormone (ACTH) and the adrenal cortex to release corticosteroids, which increase energy.

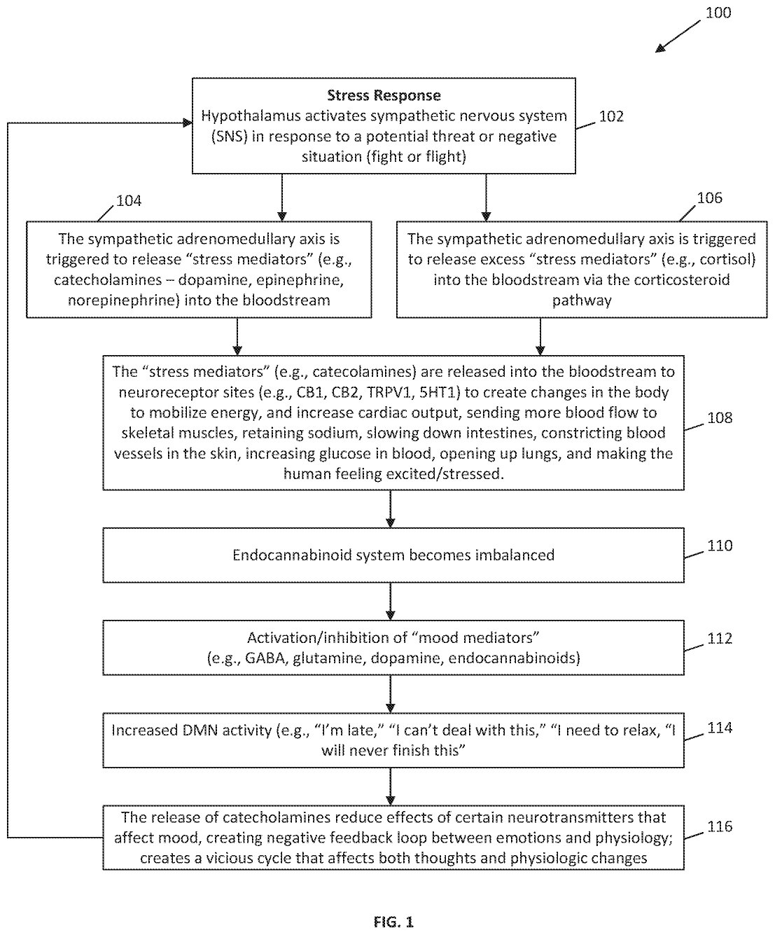

Referring to FIG. 1, the stress response process 100 begins at 102 when the hypothalamus activates the sympathetic nervous system in response to a potential threat or negative situation (“fight or flight”). As the stress response is triggered and the body's sympathetic nervous system is activated, at 104 and 106, the adrenal glands release stress hormones like cortisol, while the sympathetic-adrenomedullary axis (SAM) is also triggered to release catecholamines (e.g., dopamine, epinephrine, norepinephrine). These circulate through the bloodstream and the brain. They act on neuro-receptor sites to create changes in the body to mobilize energy. This is part of the “fight or flight,” response—putting the body on alert.

At block 108 of FIG. 1, the “stress mediators” (e.g., catecholamines) are released into the bloodstream to neuroreceptor sites (e.g., CB1, CB2, TRPV1, 5HT1) to create changes in the body to mobilize energy. The immediate effects of catecholamines include: increasing cardiac output, sending more blood flow to skeletal muscles, retaining sodium, slowing down the intestines, constricting the blood vessels in the skin, increasing glucose in the bloodstream, opening up lungs, and making a person feel excited, nervous, stressed and/or on edge. The heart beats faster and directs the flow to muscles, enabling the ability to run or fight. By reducing blood flow to the skin, there may be less bleeding in case of injury. This causes faster breathing and more oxygen.

Prolonged exposure to catecholamine's can create negative psychological and physical outcomes. Prolonged release of catecholamines can reduce the effects of certain neurotransmitters that affect mood, creating a negative feedback loop between emotions and physiology. These changes can also lead to chronic inflammation of organs and the failure of adaptive systems. This can lead to behavior and quality of life changes, sleep disturbances, metabolic disturbances and cardiovascular disturbances.

The catecholamines are part of the body's parasympathetic nervous system (PNS) or the relaxation response. This calms the body's physiology and returns the body to its pre-stressed state when the perceived threat is gone.

Endocannabinoid System (ECS)—The endocannabinoid system is a biological system within all humans. Endocannabinoids, that is, anadamide (AEA) and 2-Arachidonoylglycerol (2-AG), are neurotransmitters that bind to cannabinoid receptors and cannabinoid receptor proteins, which are expressed throughout the vertebrate central nervous system (including the brain) and peripheral nervous system. The endocannabinoids are messengers synthesized by the body. The two main cannabinoid receptors are CB1 and CB2. CB1 receptors are found predominantly in the brain and nervous system, and in peripheral organs and tissues. CB1 receptors are the main target of AEA, as well as exogenous cannabinoids such as cannabidiol (CBD), cannabinol (CRN) and tetrahydrocannabinol (TI-IC) found in cannabis plants. The 2-AG endocannabinoid acts as a full agonist at both the CB1 and CB2 receptors.

As a result of stress, at block 110, a person's ECS can become imbalanced. People in a chronic stress state have been found to have low levels of the natural endocannabinoids AEA and 2-AG. These chemical messengers, which are naturally found in the brain and tissues of the body, act as a bridge between your body and brain, helping regulate multiple functions. For example, cannabidiol (CBD) oil extracted from a cannabis plant upregulates or produces more receptors for endocannabinoids to bind. That could help in terms of calming stress and modulating the negative impact of stress on brain function as well as immunity and other functions. Essentially, the ECS provides the fine-tuning or modulation of the stress pathways to decrease input and induce the relaxation response.

One theory is that the endocannabinoids, AEA and 2-AG, act as “gatekeepers” to keep the stress response and high emotional loads at bay and improve recovery to pre-stress levels once the stress burden is reduced to baseline. Cannabinoids are thought to rebalance or augment responses to stress. Moreover, there is evidence from preliminary human studies that disrupting endocannabinoid signaling and regulation has an important impact on the hypothalamic-pituitary-adrenal axis (HPA-axis) by increasing signs of anxiety and depression. The crosstalk between the immune system, cytokine signaling, and the nervous system influencing behavior, anxiety and mood has been proposed by numerous preclinical studies.

A number of recent studies have demonstrated the role of CB2 receptors classically associated with the immune system in anxiety and depression-related behavior in animal models and human clinical studies. In this context, it is interesting to note that a balanced ECS appears to be pivotal to a healthy stress response and mitigating fear, anxiety, and panic that tend to accumulate with dysfunctional responses to stress.

At block 112, as a part of the stress response, “mood mediators” become activated and/or inhibited. The mood mediators include, for example, gamma aminobutyric acid (GABA), glutamine, dopamine, endocannabinoids, etc. This leads to, at block 114, increased default mode network (DMN) activity. The DMN is a large scale brain network of interacting brain regions known to have activity highly correlated with each other and distinct from other networks in the brain. The increased DMN activity can produce thoughts such as “I'm late,” “I can't deal with this,” “I need to relax” and “I will never finish this.”

At block 116, the release of catecholamines can reduce the effects of certain neurotransmitters that affect mood, thereby creating a negative feedback loop between emotions and physiology. This creates a vicious cycle that affects both thoughts and physiologic changes.

In other words, a stimulus perceived by the central nervous system, including the brain, increases hypothalamus activity and activates the sympathetic nervous system (SNS). The SAM is triggered to release catecholamines to increase adrenaline (epinephrine) and noradrenaline (norepinephrine) in the blood stream resulting in an increase in cardiovascular activity and an increase in stress. Further activation occurs in the endocrine system via the hypothalamic-pituitary adrenocortical axis (HPA-axis), which leads to further release of hormones like cortisol and other transmitters that increase heart rate, blood pressure, etc. causing feelings of excitement and heart fluttering. An increase in stress mediators (e.g., catecholamines, glucocorticoids) released into the bloodstream to neuro-receptor sites creates changes in the body to mobilize energy. Continued activation also alerts the immune system, which is placed on high alert, causing the immune system to produce various chemicals that lead to inflammation resulting in increased susceptibility to colds/infection. Binding and activation of key receptors, CB1, CB2, TRPV1 and 5HTP1, takes place and within the CNS there is an activation/inhibition of the “mood mediators,” GABA, glutamate, dopamine, serotonin and endocannabinoids. All of this activity leads to an imbalance across these three (3) systems: the CNS, the endocrine system and the immune system as they can become depleted of various chemicals and transmitters that balance things out. This creates a vicious cycle that affects both thoughts and physiologic changes.

Relaxation Response—The counterpart to the “fight-or-flight” response, the relaxation response, occurs when the body is no longer in perceived danger, and the autonomic nervous system functions to return to normal. Simply put, the relaxation response is the opposite of the body's stress response—an “off switch” to the body's tendency toward fight-or-flight.

During the relaxation response, the body moves from a state of physiological arousal, including increased heart rate and blood pressure, slowed digestive functioning, decreased blood flow to the extremities, increased release of hormones like adrenaline and cortisol, and other responses preparing the body to fight or run, to a state of physiological relaxation, where blood pressure, heart rate, digestive functioning, and hormonal levels return to their normal state.

During acute stress, this response occurs naturally. Historically, this worked well when the stress response was triggered, somewhat rarely, and meant fast-moving physical threats like predators. In modern times, however, as the stress response is triggered multiple times throughout the day, the relaxation response does not always have a chance to naturally follow, creating an imbalance and a persistent stress response. For example, in times of chronic stress, the body is in a constant state of physiological arousal over perceived threats that are numerous and not life-threatening, and the body's relaxation response does not always have time to kick in before the next stressor hits. This decreases immunity and increases negative emotional consequences such as anxiety and burnout.

In situations like these, the relaxation response can be induced through techniques that relax the physical or physiological body, or the mind—if both can be relaxed simultaneously, it is even more effective. With all of today's modern technology and increased demands on an individual's time and heightened expectations, stress and anxiety has become more of an issue. There have been many approaches for reducing stress, either pharmacologically, or via cognitive approaches such as meditation, breathing, yoga, or exercising. There has not been a single type of treatment that can address the continuum of the stress response, and thus, what is needed, is a treatment that provides a holistic approach, leveraging and affecting multiple pathways involved in stress.

Homeostasis—There are two branches of the nervous system—the sympathetic nervous system (SNS) and the parasympathetic nervous system (PNS). Their primary function is to maintain what is referred to as homeostasis (i.e., balance). In times of stress, the sympathetic nervous system is activated (i.e., the “flight or flight” response discussed above) causing the heart to beat faster, and the person to feel nervous and on edge, for example, as if consuming too much coffee. Fight or flight is a primitive response that is activated when the body (i.e., the brain) senses danger or something that may cause worry. The response is communicated by neurotransmitters and activated hormones that kick off a cascade of events to prepare for fight or flight.

Homeostasis is achieved when the two nervous system branches are in balance. Once a stressor is removed or fear subsides, a person begins to feel back in control due to the parasympathetic system. The parasympathetic nervous system counteracts the sympathetic nervous system, releasing neurotransmitters that induce a more relaxed state, lowering the heart rate, blood pressure and jitters. Activation of the parasympathetic nervous system to counteract the sympathetic nervous system causes the “relaxation response” discussed above. Breathing and aromatherapy can induce the relaxation response and are helpful to calm the body in times of stress. The parasympathetic nervous system helps return the two systems back into homeostasis—a rebalancing or resetting of those two systems.

Allostasis—Two additional systems in the body that are also activated by the stress response are the immune system and the endocrine system. As discussed above, these two systems communicate closely with the nervous systems and, in times of stress, the immune and endocrine systems also respond and can become imbalanced. During time of stress, the immune system can become depressed making it easier for a person to catch a cold. The Endocrine system, which regulates various hormones including cortisol (a.k.a. the stress hormone) also can become imbalanced. When homeostasis is achieved together with balance of the immune and endocrine systems, this is referred to as “allostasis.”

### Dosage Forms

Dosage forms and kits according to embodiments herein, target all three pathways, the nervous system, endocrine system and immune system, to help bring the body back into allostasis. Dosage forms, dietary supplements and/or methods according to embodiments herein target all three of these systems to help support the body in reaching allostasis thereby lessening stress.

According to embodiments herein, dosage forms can include a sensory disrupter, a stress-buster and a calming reliever. Suitable sensory disrupters include, but are not limited to, a terpene, linalool, limonene, caryophyllene (also has warming properties), cooling agents (e.g., menthol, menthol-like, etc.), warming agents (e.g., cinnamon, black pepper, cardamom, clove bud, ginger, juniper berry, marjoram, rosemary and oils thereof), any suitable colors to stimulate sight (e.g., to provide a relaxed and/or happy feeling), agents that stimulate taste (e.g., aromatic oils as described herein, suitable food products, pop rock-like, etc.), and components that stimulate sound (e.g., popping as in pop rocks, crinkling, wind, rain, birds, ocean, music, classical music, jazz music, relaxation app, etc.) and combinations thereof. Suitable stress-busters include, but are not limited to, L-arginine, L-theanine, lysine, glycine, gamma amino butyric acid (GABA), magnesium, citi-choline, inositol, apigenin and combinations thereof. Suitable calming relievers include, but are not limited to, cannabidiol, lemon balm, passionflower, ashwagandha, valerian root extract and combinations thereof.

According to various embodiments, the dosage form can be formulated for any suitable route of administration known to those of ordinary skill in the art. In embodiments, the dosage form can be formulated for a route of administration selected from oral, parenteral, subcutaneous, intravenous, intramuscular, intraperitoneal, transdermal, transmucosal (e.g., via the transurethral or rectal routes), sublingual, buccal, gingival, rectal, subcutaneous, transpulmonary, topical, nasal, inhalation and combinations thereof. According to embodiments, dosage forms as described herein may be in the form of a tablet, troche, lozenge, a plurality of units (as described in more detail below), hard capsule, soft capsule, buccal tablet, buccal film, sublingual tablet, sublingual film, transdermal patch, topical gel, cream, oil or ointment, liquid (e.g., a liquid shot), etc. In embodiments, the dosage form is a shot having a volume of about 1 ml to about 100 ml, about 2 ml to about 75 ml, about 5 ml to about 50 ml, or about 10 ml to about 25 ml.

According to embodiments, dosage forms as descried herein can be formulated to have quick or rapidly dissolving properties that would provide an oral sensation of dissolving quickly within the oral cavity. In embodiments, the dosage forms can be formulated to provide rapid delivery (e.g., immediate release) via sub-lingual application incorporating the various components as described herein. In embodiments, oral dosage forms can employ ingredients that produce an effervescent reaction upon placement into liquid, such as water or juice. In yet further embodiments, oral dosage forms described herein can be formulated into a gelatin gummy form. Such “gummies” can incorporate various components that invoke one or more of the following sensations: a cooling or heating sensation within the oral cavity, and bitter, sour and/or sweet taste upon ingestion via oral cavity. In yet further embodiments, dosage forms can be formulated for delivery via a “tea-bag” that containing ingredients as set forth herein. This “tea-bag” form can also employ aromatic oils are natural oils such as plant terpenes to stimulate the olfactory system, thus providing a sensory disruption to the sympathetic nervous system.

In embodiments, the dosage form can comprise a plurality of units. The plurality of units can include, but are not limited to, particles, powder, granules, beads, microspheres, micro-tablets, microcapsules, extrudates and combinations thereof. In certain embodiments, the plurality of units are in the form of micro-tablets. The micro-tablets can have any suitable shape known to those of ordinary skill in the art, for example, the micro-tablets can be circular, cylindrical, pyramidal, toroidal and combinations thereof. The micro-tablets can be formed from compressed powder, beds, microspheres and/or granules or can be extrudates. Each of the plurality of units can have a mean size of about 0.1 μm to about 5 mm, or about 1 μm to about 3 mm, or about 50 μm to about 1 mm. In embodiments, the micro-tablets can have a mean size of about 0.5 mm to about 5 mm.

According to various embodiments, the plurality of units can include a first plurality of units and a second plurality of units. The first plurality of units may be a different color than the second plurality of units. For example, the first plurality of units may be white and the second plurality of units may be orange or purple. The color can be selected to have a soothing effect and may be selected from shades of green (e.g., sage green), blue (e.g., baby blue, slate blue, etc.), purple (e.g., lavender), white, grey (e.g., soft grey), pink, yellow and orange (e.g., mandarin orange). In embodiments, the plurality of units can be divided into, two, three, four and so on. Each of the plurality of units can have a different color. The plurality units can include natural pigments to provide the desired color. Such pigments can be food colorants and may include, but are not limited to, flavonoids, spirulina, beets, orange carrots, pumpkins, black carrots, sweet potatoes, elderberries, tomatoes, blueberries, grapes, safflower and combinations thereof.

According to various embodiments, the plurality of units can be formulated to provide a controlled release of one or more component therein. The terms “controlled-release,” “extended-release” or “sustained release” are interchangeable and are defined for purposes of the present disclosure as the release of one or more component (e.g., a sensory disrupter, a stress buster, a calming reliever, etc.) at such a rate that blood (e.g., plasma) concentrations are maintained within the therapeutic range, but below toxic concentrations, over a period of time of at least about 1 hour or longer, at least about 2 hours or longer, at least about 3 hours or longer, at least about 4 hours or longer, at least about 5 hours or longer, or at least about 6 hours or longer, or at least about 10 hours or longer, or at least about 12 hours or longer or at least about 24 hours or longer. Preferably, a controlled release dosage form can provide one, two, three or four times daily dosing.

In embodiments, the plurality of units can be formulated to provide an immediate release of one or more component therein. The term “immediate release” is defined for the purposes of the present disclosure as the release of one or more component (e.g., a sensory disrupter, a stress buster, a calming reliever, etc.) into the air, mouth or in the gastrointestinal contents with no delay or prolongation of dissolution or absorption, smell or taste of the one or more component.

In embodiments, the dosage form may comprise a pharmaceutically acceptable capsule. In embodiments, at least one of a sensory disrupter, a stress buster and a calming reliever can be within the pharmaceutically acceptable capsule. In embodiments where the dosage form comprises a plurality of units, the plurality of units can be contained within the pharmaceutically acceptable capsule. The capsule may be any suitable capsule known to those of ordinary skill in the art including a hard shell capsule, a soft shell or a soft gelatin capsule. The pharmaceutically acceptable capsule can be formed of a mixture of natural oils and components. In embodiments, the pharmaceutically acceptable capsule comprises a sensory disrupter, for example, at least one aromatic oil (e.g., a natural oil) as described in embodiments herein. In embodiments, the aromatic oil comprises at least one of mint oil, lavender oil and citrus oil. Suitable components used to form the capsule shell can include, but are not limited to, gelatin and a physiologically acceptable plasticizer such as glycerol (e.g., the gelatin forming a matrix for the plasticizer). The capsule shell composition can include about 15% to about 30% by weight of gelatin and about 30% to 45% by weight of the plasticizer. The further component is normally a potato starch acetate, another starch derivative, starch itself or mixtures thereof. According to embodiments, the capsule shell can be clear or translucent.

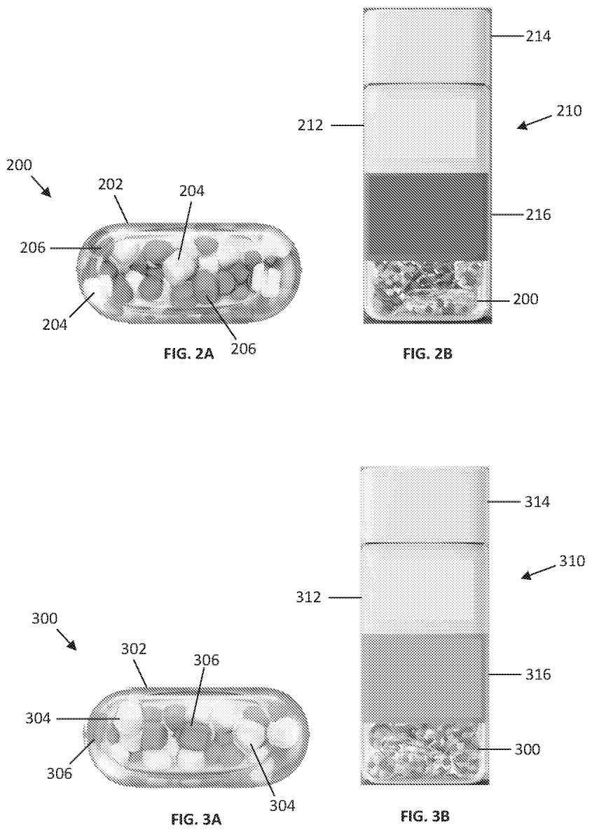

Examples of dosage forms according to various embodiments herein are shown in FIGS. 2A and 3A. As shown, the pharmaceutically acceptable capsule 202, 302 may be clear or translucent with micro-tablets contained therein. A first plurality of the micro-tablets 204, 304 can be a white color and the second plurality of the micro-tablets 206, 306 can be a different color, for example, purple 206 or orange 306. The appearance of the dosage form may itself provide a sensory disruption to someone experiencing stress where the sight of a soothing color and unique look can help in rebalancing the body's systems to achieve allostasis.

According to embodiments, the pharmaceutically acceptable capsule comprises a coating on a surface thereof. The coating can include a sensory disrupter, for example, at least one aromatic oil. Suitable aromatic oils include, but are not limited to a plant terpene, lavender oil, citrus oil, chamomile oil, rose oil, hyssop oil, mint oil, peppermint oil, spearmint oil, wintergreen oil, clary sage oil, rosemary oil, ylang ylang oil, myrrh oil, vetiver oil, lemongrass oil, orange oil, grapefruit oil, lemon oil, frankincense oil, helichrysum oil, cedarwood oil, basil oil, melaleuca oil, arborvitae oil, patchouli, clove oil, cassia oil, oregano oil, rosemary oil, thyme oil, mango oil, juniper oil, cinnamon oil, pepper oil, lilac oil, nutmeg oil, cumin oil, pine oil, parsley oil, orchid oil and cannabis oil. In embodiments, the coating comprises at least one of mint oil, lavender oil and citrus oil. Suitable plant terpenes include, but are not limited to myrcene, pinene, caryophyllene, limonene, terpinolene and combinations thereof.

According to various embodiments, the dosage form provides an immediate release of at least one of the sensory disrupter, the stress-buster and the calming reliever. In embodiments, the dosage form provides a controlled release of at least one of the sensory disrupter, the stress-buster and the calming reliever. In further embodiments, the dosage form provides an immediate release of at least one of the sensory disrupter, the stress-buster and the calming reliever and a controlled release of at least one of the sensory disrupter, the stress-buster and the calming reliever. In certain embodiments, the dosage form, for example, as shown in FIGS. 2A and 3A comprises a sensory disrupter (e.g., an aromatic oil) coated onto the capsule or formulated within the capsule shell material. The sensory disrupter can be in an immediate release form so that upon opening a package or container housing the dosage form therein a human can smell the aromatic oil, upon placing the dosage form in his/her mouth can taste the oil and within seconds or minutes of swallowing the dosage form can feel a warming and/or cooling sensation.

According to certain embodiments, the dosage form may be free of certain components, for example, depressants, narcotics, supplements, etc. that may cause sleep or drowsiness. The dosage form may alternatively or additionally be free of components, for example, stimulants, amphetamines, supplements, etc. that may cause insomnia or jitters. In embodiments, the dosage form is free of at least one of melatonin, diphenhydramine, caffeine, cannabinoids, cypress oil, eucalyptus oil, tea tree oil, rose oil, sandalwood oil or a drug (e.g., a substance intended for use in the diagnosis, cure, mitigation, treatment, or prevention of disease).

According to further embodiments, disclosed herein is a dosage form, comprising a nervous system component, an immune system component and an endocrine system component. Suitable nervous system components include, but are not limited to gamma amino butyric acid, valerian root, 5-hydroxytryptophan, L-theanine, passionflower, lemon balm and combinations thereof. Suitable immune system components include, but are not limited to Ahi flower oil, MCT oil, Vitamin D3, an aromatic oil and combinations thereof. Suitable endocrine system components include, but are not limited to magnolia extract, phaelladendron extract, ashwagandha and combinations thereof.

As discussed above, suitable aromatic oils include, but are not limited to a plant terpene, lavender oil, chamomile oil, rose oil, hyssop oil, mint oil, peppermint oil, spearmint oil, wintergreen oil, clary sage oil, rosemary oil, ylang ylang oil, myrrh oil, vetiver oil, lemongrass oil, orange oil, grapefruit oil, lemon oil, frankincense oil, helichrysum oil, cedarwood oil, basil oil, melaleuca oil, arborvitae oil, patchouli, clove oil, cassia oil, oregano oil, rosemary oil, thyme oil, mango oil, juniper oil, cinnamon oil, pepper oil, lilac oil, nutmeg oil, cumin oil, pine oil, parsley oil, orchid oil, cannabis oil and combinations thereof.

As discussed above, the dosage form can be formulated for any suitable route of administration known to those of ordinary skill in the art. In embodiments, the dosage form can be in the form of a plurality of units, as discussed above, contained within a soft shell capsule, also as discussed above and as shown in FIGS. 2A and 3A. The dosage form may include a controlled release component, an immediate release component or a combination of controlled release and immediate release components as described above. In embodiments, the dosage form is free of at least one of melatonin, diphenhydramine, caffeine, cannabinoids, cypress oil, eucalyptus oil, tea tree oil, rose oil, sandalwood oil or a drug (e.g., a substance intended for use in the diagnosis, cure, mitigation, treatment, or prevention of disease).

Dosage forms according to embodiments herein may include a coating on a surface thereof. The coating can include a sensory disrupter, for example, at least one aromatic oil. Suitable aromatic oils include, but are not limited to a plant terpene, lavender oil, citrus oil, chamomile oil, rose oil, hyssop oil, mint oil, peppermint oil, spearmint oil, wintergreen oil, clary sage oil, rosemary oil, ylang ylang oil, myrrh oil, vetiver oil, lemongrass oil, orange oil, grapefruit oil, lemon oil, frankincense oil, helichrysum oil, cedarwood oil, basil oil, melaleuca oil, arborvitae oil, patchouli, clove oil, cassia oil, oregano oil, rosemary oil, thyme oil, mango oil, juniper oil, cinnamon oil, pepper oil, lilac oil, nutmeg oil, cumin oil, pine oil, parsley oil, orchid oil and cannabis oil. In embodiments, the coating comprises at least one of mint oil, lavender oil and citrus oil. Suitable plant terpenes include, but are not limited to myrcene, pinene, caryophyllene, limonene, terpinolene and combinations thereof.

Dosage forms according to embodiments herein can include at least one additional active ingredient (e.g., a drug). Suitable additional active ingredients include, but are not limited to an anti-anxiety drug, an antidepressant, a prodrug thereof, a cannabinoid, dronabinol, a pharmaceutically acceptable salt thereof and combinations thereof. Suitable anti-anxiety drugs include, but are not limited to a central nervous system depressant, a benzodiazepine, buspirone, buproprion, a prodrug thereof, a pharmaceutically acceptable salt thereof and combinations thereof. Suitable benzodiazepines include, but are not limited to alprazolam, chlordiazepoxide, clonazepam, diazepam, lorazepam, a prodrug thereof, a pharmaceutically acceptable salt thereof and combinations thereof.

Suitable central nervous system depressants include, but are not limited to a barbiturate, a prodrug thereof, a pharmaceutically acceptable salt thereof and combinations thereof. According to embodiments, the barbiturate includes at least one of secobarbital, butabarbital, mephobarbital, pentobarbital, phenobarbital, amobarbital, a prodrug thereof, a pharmaceutically acceptable salt thereof and combinations thereof. Suitable antidepressants include, but are not limited to a selective serotonin reuptake inhibitor (S SRI), a selective noradrenaline reuptake inhibitor (SNRI), a tricyclic, a monoamine oxidase inhibitor (MAOI), a beta-blocker, a sedative, a prodrug thereof, a pharmaceutically acceptable salt thereof and combinations thereof. According to embodiments, the SSRI can include at least one of citalopram, escitalopram, fluvoxamine, cerixamine, fenfluramine, paroxetine, dapoxetine, dapoxetine, fluoxetine, ifoxetine, vortioxetine, sertraline, vilazodone, cyanodothiepin, nefazodone, a prodrug thereof, a pharmaceutically acceptable salt thereof and combinations thereof.

Suitable SNRIs include, but are not limited to venlafaxine, desvenlafaxine, milnacipran, duloxetine, levomilnacipran, reboxetine, desipramine, maprotiline, lofepramine, mirtazapine, oxaprotilin, fezolamine, atomoxetine, bupropion, mianserin, duloxetine, a prodrug thereof, a pharmaceutically acceptable salt thereof and combinations thereof. Suitable tricyclics include but are not limited to clomiprmamine, imipramine, a prodrug thereof, a pharmaceutically acceptable salt thereof and combinations thereof. Suitable MAOIs include, but are not limited to socarboxazid, phenelzine, selegiline, tranylcypromine, a prodrug thereof, a pharmaceutically acceptable salt thereof and combinations thereof. According to embodiments, the beta-blocker can include at least one of propranolol, atenolol, a prodrug thereof, a pharmaceutically acceptable salt thereof and combinations thereof.

In yet further embodiments, disclosed herein are dosage forms, comprising the following ingredients: vitamin D3, Ahi flower, MCT oil, L-Theanine, 5-hydroxytryptophan, valerian root, a mixture of phellodendron amurense and magnolia officinalis, gama amino butyric acid, ashwagandha, beta caryophyllene, citrus oil and mint oil. According to embodiments, the dosage form is a pharmaceutically acceptable capsule comprising a combination of natural oils and components as described herein. Optionally, the capsule provides an immediate release of the natural oils and components. In embodiments, the capsule is translucent or clear. The ingredients can be in the form of a plurality of units, for example, the ingredients may be prepared as a granulation or powder and formed into compressed micro-tablets or extrudates. In embodiments, a first plurality of the plurality of units is a different color from a second plurality of the plurality of units, and optionally the plurality of units provide a controlled release of the ingredients. According to various embodiments, the dosage form comprises at least one additional active ingredient as described above. In embodiments, the dosage form is an evening formulation to be taken during the day in anticipation of stress to support the body during its stress response enabling it to balance its systems effectively to achieve allostasis. In embodiments, Vitamin D3 Oil can be present in an amount of about 5 mcg/unit to about 100 mcg/unit (i.e., per capsule, per tablet, per two capsules, per shot, etc.), Ahi flower Oil can be present in an amount of about 100 mg/unit to about 500 mg/unit, MCT Oil can be present in an amount of about 50 mg/unit to about 275 mg/unit, L-Theanine can be present in an amount of about 25 mg/unit to about 250 mg/unit, 5-Hydroxy tryptophan (5-HTP) (Griffonia simplicifolia), can be present in amount of about 2.5 mg/unit to about 10 mg/unit, Valerian Root Officinalis CO₂ extract can be present in an amount of about 0.5 mg/unit to about 10 mg/unit, a mixture of magnolia officinalis and phellodendron amurense can be present in an amount of about 25 mg/unit to about 250 mg/unit, GABA can be present in an amount of about 25 mg/unit to about 250 mg/unit, Ashwagandha can be present in an amount of about 25 mg/unit to about 125 mg/unit, and Beta Caryophyllene can be present in an amount of about bout 0.5 mg/unit to about 10 mg/unit, where a unit is, for example, 1 capsule, tablet, shot, etc.

In yet further embodiments, disclosed herein are dosage forms, comprising vitamin D3, Ahi flower, MCT oil, passionflower, lemon balm, chamomile flower, valerian root, a mixture of phellodendron amurense and magnolia officinalis, gama amino butyric acid, ashwagandha, beta caryophyllene, lavender oil and mint oil. According to embodiments, the dosage form is a pharmaceutically acceptable capsule comprising a combination of natural oils and ingredients as described in embodiments above. In embodiments, the capsule provides an immediate release of the natural oils and ingredients. Optionally, the capsule is translucent or clear. The ingredients can be in the form of a plurality of units as described according to embodiments herein. In embodiments, a first plurality of the plurality of units is a different color from a second plurality of the plurality of units. The plurality of units can provide a controlled release of the ingredients. In embodiments, the dosage form can include at least one additional active ingredient according to embodiments described herein. In embodiments, the dosage form is an evening formulation to be taken at the end of the day to help rebalance and support the body in achieving allostasis and achieve restful sleep. In embodiments, Vitamin D3 Oil can be present in an amount of about 5 mcg/unit to about 100 mcg/unit, Ahi flower oil in an amount of bout 100 mg/unit to about 500 mg/unit, MCT Oil in an amount of about 50 mg/unit to about 275 mg/unit, Passionflower (Passiflora incarnata) in an amount of about 5 mg/unit to about 100 mg/unit, Lemon Balm (Melissa officinalis) in an amount of about 5 mg/unit to about 50 mg/unit, Chamomile Flower (Matricaria chamomilla) CO₂-extract (organic) in an amount of about 0.25 mg/unit to about 5 mg/unit, Valerian Root officinalis CO₂ extract in an amount of about 5 mg/unit to about 10 mg/unit, a mixture of magnolia officinalis and phellodendron amurense in an amount of about 25 mg/unit to about 250 mg/unit, GABA in an amount of about 25 mg/unit to about 250 mg/unit, Ashwagandha in an amount of about 25 mg/unit to about 125 mg/unit, and Beta Caryophyllene about 5 mg/unit to about 10 mg/unit, where a unit is, for example, a capsule, a tablet, a shot, etc.

In addition to the above ingredients, a dosage forms according to embodiments herein may also contain suitable quantities of other materials, e.g., diluents, lubricants, binders, granulating aids, colorants, flavorants and glidants that are conventional in the pharmaceutical art.

### Kits

According to various embodiments, disclosed herein are kits comprising dosage forms in accordance with embodiments herein. The kit can include a container and at least one dosage form removably positioned within the container. In accordance with various embodiments, the dosage form comprises at least one sensory disrupter, at least one stress-buster and at least one calming reliever as described herein. In embodiments, the dosage form comprises at least one central nervous system component, at least one immune system component and at least one endocrine system component as described herein.

According to embodiments, the container can be formed of a material that targets the sympathetic nervous system and the container itself can be a sensory disrupter. In embodiments the container can be formed of and/or contain at least one of a textured material, a smooth material, a soft material, a wood material, a bamboo material and a calming color that, upon touch, sight or both, provides a sensory disruption to a human. By focusing on the soothing container, the human can begin to reset and balance the stress response.

In embodiments, the kit further comprises an aromatic oil as described herein. The aromatic oil can be contained within a canister, sachet, film or any other suitable device that is positioned within, on and/or near the container. In embodiments, the aromatic oil can be coated on an inner surface of the container, coated on an outer surface of the container, coated on an outer surface of the dosage form, contained within the dosage form (e.g., in a capsule shell material), or a combination thereof.

Example embodiments of kits 210, 310 are shown in FIGS. 2B and 3B. As shown in FIGS. 2B and 3B, each kit 210, 310 contains at least one dosage form 200, 300. In these embodiments, an aromatic oil is coated onto and/or formulated in the material of the capsule shell 202, 302. The at least one dosage form 200, 300 is as shown and described in FIGS. 2A and 3A. Each kit 210, 310 further includes a container 212, 312 having a lid 214, 314 enabling the at least one dosage form 200, 300 to be removably contained therein. The container can include a color 216, 216, for example, that matches the colors of the dosage form 200, 300. The color can provide sensory disruption to a human viewing the kit 210, 310.

According to embodiments, the kit further includes external packaging (not shown) such that the container 212, 312 with the at least one dosage form 200, 300, respectively, is removably positioned within the external packaging. Like the container 212, 312, the external packaging can include at least one of a textured material, a smooth material, a soft material, a wood material, a bamboo material and a calming color that, upon touch, sight or both, provides a sensory disruption to a human. The external packaging may include an aromatic oil as described herein. The aromatic oil can be contained within a canister, sachet, film or any other suitable device that is positioned within and/or on the external packaging. In embodiments, the aromatic oil can be coated on an inner or outer (or both) surface of the external packaging, coated on an outer surface of the container.

According to embodiments, the kit includes instructions for using the kit to reduce at least one of stress and anxiety, for example, by supporting the body's response to stress. The instructions can direct a human to combine sensory disruption together with administering the at least one dosage form to achieve a synergistic effect that assists the body's response to stress and to achieve allostasis. In embodiments, the kit comprises instructions to a human to open the container and hold the container under the person's nose. In embodiments the instructions can direct the human to exhale then breathe in through the nose drawing breath all the way into belly—optionally, to pause for about 1 second to about 10 seconds, and then optionally to exhale from the chest, for example, to imagine the belly button touching the spine. In embodiments, the instructions can direct the human to repeat the breathing and exhaling as desired and/or optionally for a particular number of times, for example, 5, 6, 7, 8, 9, 10, 15 or 20 times. In embodiments, the instructions can direct the human to insert one dosage form into his or her mouth and/or to taste the dosage form while swallowing.

### Sensory Disrupters

A sensory disrupter is a component, composition and/or action that targets smell, sight, touch, feel and/or taste. In embodiments, sensory disrupters that target smell include, but are not limited to aromatic oils according to embodiments described herein. In certain embodiments, a sensory disrupter targeting smell comprises at least one of linalool, limonene, caryophyllene and combinations thereof. Sensory disrupters targeting sight include the visual appearance of the dosage form including, but not limited to micro-tablets within a clear capsule, a capsule within a capsule, the colors of the micro-tablets, the colors of the capsule, etc. and combinations thereof. Sensory disrupters targeting touch and feel include, but are not limited to rubbing, massaging, gels, creams, ointments, cooling, warming and combinations thereof. Sensory disrupters that target taste include, but are not limited to cooling flavors or terpenes (e.g., menthol, menthol-like, etc.), warming flavors or terpenes (e.g., caryophyllene), citrus and lemon (e.g., limonene) and lavender (e.g., linalool).

In particular, with respect to touch and feel, in people with the movement disorder cervical dystonia, a condition where various neck/shoulder muscles are chronically retracted leading to disfigurement and significant pain, the person temporarily interrupts the motor nerve inputs using a “trick” or what is referred to as a “geste atagoniste.” These tricks, which have many variations, apply a physical stimulus to and/or by the person. For example, the physical stimulus can include squeezing the wrist or arm, pressing on a muscle within the unaffected side of the neck or shoulder, which temporarily interrupts the physiologic signals to the affected muscles and/or touching a region on the person's face. Studies have shown that there is a physiologic down regulation that occurs simply by applying a sensory stimulus.

Another example of a sensory disrupter is when a toddler falls down and suddenly realizes it and begins to cry. The parent will provide a stimulus, either vocally—a soothing sound of the mother's voice, or physically—the mother picks up and holds the toddler. As a result, the sensory disrupter is enough to interrupt the toddler's current stress level triggered by the fall.

Yet another example of a sensory disrupter is of a person who suffers chronic hyperemesis syndrome (CHS)—a condition defined by symptoms including significant nausea, vomiting and abdominal pain in the setting of chronic cannabis use. These patients find temporary relief by taking multiple very-hot water showers throughout the day—a sensory component to treat a central condition. Relief of CHS symptoms with very hot water (greater than 41° C.) has highlighted a peripheral tissue receptor called TRPV1, a G-protein coupled receptor that has been shown to interact with the endocannabinoid system, but is also the only known capsaicin receptor. The result is a temporary down-regulation of the overactive autonomic nervous system.

### Stress Busters

Stress busters are components that assist the body in down-regulating catecholamine and cortisol release in the CNS and the endocrine system. Certain mediators block receptors (i.e., competitive inhibition) or bind receptors to increase dopamine, GABA, glutamine, glycine and/or decrease norepinephrine and epinephrine. Suitable stress buster components for dosage forms according to embodiments herein include, but are not limited to L. Arginine, L. Lysine, GABA, glycine, magnesium, citi-choline, inositol, apigenin and combinations thereof.

There are many reports on various herbs and components known for their anxiolytic properties. These studies have reported relief of stress, some as good, if not better than, existing pharmacologic treatments such as benzodiazepines. Other studies have found enhanced relief by combining two or more ingredients.

### Calming Relievers

Calming relievers are components that assist in down-regulating stress mediators and receptors in the ECS. Certain mediators block receptors (i.e., competitive inhibition) or bind receptors, e.g., CB1, CB2, TRPV1 and 5HT1, to increase dopamine, GABA and glycine. Suitable calming reliever components for dosage forms according to embodiments herein include, but are not limited to one or more cannabinoids (e.g., cannabidiol, cannabinol, tetrahydrocannabinol, dronabinol, etc.), caryophyllene, lemon balm, passionflower, ashwagandha and combinations thereof.

Surprisingly, and in accordance with embodiments herein, it was found that combining stimulation of the sensory branch of the PNS together with a formulation of ingredients that target multiple stress pathways within the CNS can provide an acute response and a sustained response. It was found that this combination results in a decrease in stress, while simultaneously invoking the relaxation response mediated by the ANS (i.e., “Resetting and rebalancing the systems”). Dosage forms and kits according to embodiments herein provide a synergistic approach to decreasing stress by disrupting the stress response. The synergistic approach uses sensory distraction together with stress busters and calming relievers that ultimately provide both an acute and sustained response.

### Central Nervous System Components

Notably, some components disclosed herein have the capability of targeting more than one system within the body, that is, the CNS, the immune system and/or the endocrine system and/or can be considered one or more of a sensory disrupter, stress buster or calming reliever. Such components may be listed herein as suitable ingredients for targeting more than one system.

Central nervous system components target the central nervous system as described herein to help the body in responding to stress ultimately to rebalance the body's systems involved in a stress response to achieve allostasis. Suitable central nervous system components for dosage forms according to embodiments herein include, but are not limited to, gamma aminobutyric acid (GABA), valerian root, 5-hydroxytryptophan (5-HTP), L-Theanine, passionflower, lemon balm and combinations thereof. GABA the chief inhibitory neurotransmitter that works to regulate neuronal excitability and thereby serves as a “brake” on the neural circuitry during stress. It is the brain's natural stress reliever. GABAergics are also suitable components to support the nervous system. GABAergics are anxiolytic calming agents that influence GABA pathways. Valerian (i.e., Valeriana officinalis), or aqueous root extracts, can provide anxiolytic effects, decreased restlessness, reduced somatic arousal and improved sleep. Valerian also can be used in combination with hops (Humulus lupulus) and/or lemon balm (i.e., Melissa officinalis) to enhance its effects. Hops are also suitable components for dosage forms herein. 5-HTP and its precursor, tryptophan, can elevate brain serotonin levels and enhance mood and provide a sense of well-being. Passionflower (genus Passiflora) can reduce anxiety and insomnia. Studies in mice and rats have demonstrated reduced anxiety and stress with passionflower treatment, and a human study showed that treatment with passionflower can serve as an anxiolytic in humans.

A suitable component to support the HPA-Axis includes, but is not limited to one or more corticosteroid modulators. Any suitable corticosteroid known to those of ordinary skill in the art can be incorporated into dosage forms according to embodiments herein. Suitable corticosteroid modulators include, but are not limited to ashwagandha (which is believed to have an effect on the HPA-Axis), cordyceps extract (i.e., Cordyceps sinenis), selective glucocorticoid receptor modulators (SGRMs) and combinations thereof. SGRMs are designed to engage the glucocorticoid (GC) receptor with dissociative characteristics: transactivation of genes, which is mainly responsible for unwanted effects, is less strong, while transrepression of genes, reducing inflammation, is maintained. It is expected that SGRMS, thus have a better efficacy/safety balance than GCs.

According to embodiments, adaptogens are suitable components for dosage forms according to embodiments herein to target the CNS and support a human's response to stress. Adaptogenic herbs can be helpful for adaptogenic recovery to tolerate physical stress and immune support. The can help boost physical and mental energy. Adaptogens provide balance in the face of stress.

A suitable adaptogen includes, but is not limited to standardized ashwagandha root extract. One of India's most revered herbs, ashwagandha, is useful for its ability to balance, energize, and rejuvenate. Sensoril® branded ashwagandha is supported by numerous clinical studies to validate its benefits. Another suitable adaptogen includes a Chinese tree bark extract in combination with magnolia (i.e., Magnolia officinalis) and phellodendron (i.e., Phellodendron amurense) barks. A Chinese herbal supplement, used for more than 1,500 years, has been clinically proven to support normal levels of adrenal hormones for a healthful reaction to everyday stress, and to quiet the HPA axis without causing drowsiness.

L-Theanine is another suitable adaptogen to support the body during stress. This amino acid is naturally produced by the human body and found in small amounts in green tea and certain mushrooms. L-theanine competes with the human body's excited neurotransmitters, which results in balanced mood and mental focus, without causing drowsiness. One known source of L-Theanine is TheaKalm, pure L-Theanine supplement (i.e., green tea Extract) with a dosage of 200 mg/serving. The adaptogen eleuthero is also suitable to support the body's response to stress. Eleuthero is a powerful adaptogen that can strengthen tolerance to stress and promote stamina and overall health. Like other adaptogens, eleuthero primarily works by influencing the hypothalamus, a region of the brain that controls metabolism and energy (thyroid), the reaction to stress (adrenals), and the reproductive functions (testes/ovaries). By balancing the hypothalamic-pituitary-adrenal axis (HPA axis), eleuthero has been shown in randomized clinical trials to help strengthen tolerance to various types of stress, promote stamina, and reduce fatigue.

Another suitable adaptogen for dosage forms according to embodiments herein is Chinese skullcap extract (i.e., Scutellaria baicalensis). Scutellaria baicalensis can be a potent synergist to enhance the benefit of other herbs when consumed concurrently. Skullcap is a naturally occurring flavonoid isolated from Scutellaria baicalensis and can compete with benzodiazepines for binding to GABAa receptors expressed in Xenopus oocytes.

Reishi mushroom extract (i.e., Ganoderma lucidum) is another suitable adaptogen as a component of dosage forms according to embodiments herein. Reishi mushroom is an adaptogenic mushroom that improves stamina, supports immune system functions, and promotes a calm state of mind. Reishi mushroom is also neuro-supportive, liver supportive, and supports aerobic physical activity. Cordyceps extract (i.e., Cordyceps sinensis) is another adaptogenic mushroom suitable as a central nervous system component. Cordyceps extract promotes stress resistance, supports immune function, supports adrenal function and improves aerobic activity.

Another adaptogen suitable as a central nervous system component is Rehmannia extract (Rehmannia glutinosa). Rehmannia extract is an herb in traditional Chinese medicine useful as an adrenal tonic. This adaptogenic root supports adrenal function and supports a relaxed state of mind. Chamomile (i.e., Matricaria recutita) is another suitable adaptogen as a central nervous system component. Aqueous extracts of German chamomile (i.e., Matricaria recutita) and hops (Humulus lupulus) can increase GABA levels in rat brain homogenate by inhibiting GABA transaminase.

Components that target serotonin/5HT pathways are suitable central nervous system components that can provide a sense of calm. Griffonia simplicifolia is suitable as a central nervous system component. Seeds from this woody shrub are rich in 5-HTP, an amino acid dietary supplement which can elevate the levels of the neurotransmitter, serotonin in humans. Magnolia bark/extract and Phellondendron bark/extract are also suitable central nervous system components. These barks/extracts have been shown to be anxiolytic in humans in one clinical study which recommends use in premenopausal women. Another suitable central nervous system component is St. John's Wort. St. John's Wort can provide similar effects as the prescription drug Prozac. Other suitable central nervous system components include Proloftin™, which contains phosphatidyl serine, L-theanine, magnolia bark, rhodiola and beta-sitosterol, Seditol™, a proprietary blend of Magnolia officinalis and Ziziphus spinose in pill form that can be taken for relaxation, and Relarian™, a blend of aqueous valerian root extract, L-theanine, 5-HTP and GABA.

Suitable central nervous system components that target the endocannabinoid system (ECS) are hemp seed oil, natural cannabinoids (e.g., CBD, CBG, CBN, THC, THCV, CBDV), synthetic cannabinoids (e.g., dronabinol), plant terpenes, flavonoids and combinations thereof. According to embodiments, dosage forms as described herein can include cannabidiol as a central nervous system component, alone or in combination with one or more other cannabinoid, for example, tetrahydrocannabinol, which may provide a synergistic effect in balancing the ECS. In embodiments, dosage forms according to embodiments herein can comprise dronabinol as a central nervous system component.

### Immune System Components

Immune system components target the body's immune system as described herein to help the body respond to stress and ultimately rebalance the body's systems involved in a stress response to achieve allostasis. Suitable immune system components for dosage forms according to embodiments herein include, but are not limited to, Ahi flower oil, MCT oil, any other single sourced plant-based omega-3 oils, fish oil (e.g., containing omega-3 oils), structured lipids in the form of docosahexaenoic acid (DHA) and/or eicosapentaenoic acid (EPA), vitamin D3, limonene (e.g., from lemon balm), beta caryophyllene and combinations thereof.

Ahi flower oil is an omega-3 polyunsaturated fatty acid (PUFAs) known to play critical roles in neuronal cell function as well as inflammatory and immune reactions involved in neuropsychiatric disease states. Omega-3 oils are useful to modulate inflammation and support a strong immune system. MCT Oil is a medium chain triglyceride that can be taken for weight loss, appetite control, extra energy for exercise and inflammation.

Vitamin D3 supports antioxidant and anti-inflammatory activity, which is known to preserve the function of immune cells against homeostatic disturbances caused by oxidative stress and inflammation. While the amount of vitamin D3 included in dosage forms according to embodiments herein can be less than the recommended daily allowance (RDA) and serving sizes used in clinical trials, the combinatory effect of vitamin D3 and other ingredients may support immune function, for example, when taken with a healthy diet and exercise.

Beta caryophyllene is a phytochemical naturally found in many edible plants such as basil, lemon balm, clove and hemp. Through selective binding to CB1 and CB2 receptors, beta-caryophyllene may exert anti-inflammatory and stress-balancing activity. Though the amount of beta-caryophyllene include in dosage forms according to embodiments herein may be higher (e.g., slightly higher or 1%, 2%, 5%, 10% higher) than amounts in various studies, scientific evidence is supportive of its safe consumption in healthy adults.

Lemon balm is a lemon scented herb containing limonene and is useful to reduce anxiety, stress and to aid in sleep. Luteolin, a flavonoid of lemon balm, has calming and anti-inflammatory effects when administered both topically and orally, although the exact mechanism of action is uncertain. Recent in vitro research suggests that low amounts of lemon balm treatment (e.g., 100 mg) may modulate the central nervous system through interacting with GABA receptors to enhance mood states. A small amount of lemon balm in dosage forms according to embodiments herein is safe and may elicit calming and immune-enhancing effects.

### Endocrine System Components

Endocrine system components target the body's endocrine system as described herein to help the body respond to stress and ultimately rebalance the body's systems involved in a stress response to achieve allostasis. Suitable endocrine system components for dosage forms according to embodiments herein include, but are not limited to, magnolia bark extract, Phellodendron amurense bark extract, ashwagandha and combinations thereof.

Magnolia bark extract, Phellodendron amurense bark extract have shown anxiety-reducing properties in animal models. The bark of M. officinalis is useful as an antioxidant and detoxifier and has relaxing potential. One to two servings, daily, may be effective in reducing stress and supporting relaxation in healthy individuals with anxiety, for example, when taken along with a healthy diet and exercise.

Ashwagandha, native to India and the Middle East, is useful in Ayurvedic medicine for its calming, antidepressant, and sleep promoting effects. Also classified as an adaptogen, which is a substance that helps the body withstand environmental stressors, thereby promotes resilience to stress. The phytochemicals in ashwagandha counteract the negative effects of stress by increasing levels of GABA signaling and preventing overstimulation of the brain, similar to the activity of L-Theanine. Clinical evidence supports the use of ashwagandha extracts for a variety of stress-related symptoms, even at low amounts of 125 mg, for example, as included in dosage forms as described herein.

### Methods of Preparation

Dosage forms according to various embodiments described herein can be prepared using any known method known to those of ordinary skill in the art. Such methods include, but are not limited to wet granulation, dry granulation, compression, extrusion, encapsulation, and any other suitable methods known to those of ordinary skill in the art to prepare dosage forms as described herein (i.e., including gummies, topical formulations, liquids, etc.). According to embodiments, one or more ingredients of the dosage forms can be prepared in a matrix in the form of particles, a powder, granules, beads, microspheres and combinations thereof, For example, in one embodiment, granules can be formed from at least one stress buster component and at least one calming reliever component. The granules of each component can be combined into a matrix. Additional ingredients can be added to the matrix as desired. In embodiments, the matrix can be compressed and shaped to form a tablet or a micro-tablet. In further embodiments, the matrix can be converted into an extrudable form and then extruded to form extrudates.

The granules can be formed by any procedure known to those of ordinary skill in the art. For example, the granules may be formed by wet granulation with water or dry granulation, in an embodiment of a wet granulation process, at least one stress buster component and at least one calming reliever component are weighed, sifted and mixed (excluding a lubricant), optionally with one or more excipient such as a bulking agent, filler, diluent and disintegrant, in a powder mixer, The ingredients can be mixed using a planetary bowl mixer, ribbon/trough mixer, rotating drum mixer or high-speed mixer until a uniform powder mix (i.e., a matrix) is achieved. The mixing efficiency can be enhanced by the use of powders that have similar average particle size, although this is often not the case in many mixing operations.

Suitable diluents include, but are not limited to lactose, microcrystalline cellulose, starch, powdered sucrose, mannitol, fructose, sorbitol, calcium phosphate and calcium sulphate. Diluents are usually selected based on the manufacturer's experience with the material, its relative cost, and its compatibility with the drug and other excipients. Suitable disintegrants include, but are not limited to croscarmellose, sodium starch glycolate, sodium carboxymethylcellulose, polyvinylpyrrolidone (PVP), crospovidone, cation exchange resins, corn and potato starches, alginic acid and other materials that counteract the effect of binders and the physical forces of compression used in forming the tablets.

The wet granulation method can further include preparing a damp mass. In embodiments, the amount of water added during the wet granulation can be about 1.5 to about 5 times, or about 1.75 to about 3,5 times the dry weight of the matrix, The binder solution can be mixed with the powder mixture to form an adhesive mass, which can be granulated. The amount of binding agent used as well as the quantity of fluid required to form a damp and coherent mass is known to those of ordinary skill in the art. The resulting binder-powder mixture should compact when squeezed in the hand. The use of insufficient binder tends to poor adhesion, capping and soft tablets. Excessive binder solution yields hard tablets with slow disintegrating properties. Suitable granulating agents are solutions of povidone, an aqueous preparation of cornstarch, molasses, methylcellulose, hydroxypropyl methylcellulose, carboxymethylcellulose, glucose solution and microcrystalline cellulose. Suitable dry binders or nonaqueous solution can be used for substances that are adversely affected by aqueous solution. Colorants or flavoring agents can be added to the binding agent to prepare a granulation with an added feature.

The dampened powder can be wet screened into pellets or granules. The wet massed powder blend can be screened using a 6- to 12-mesh screen to prepare wet granules. This may be done by hand or with suitable equipment that prepares the granules by extrusion through perforations in the apparatus. The granules formed are spread evenly on trays and dried in an oven.

The screened moist granules are then dried in an oven at a controlled temperature not exceeding 55° C. to a consistent weight or constant moisture content. The drying temperature and the duration of drying process depend on the nature of the active ingredient and the level of moisture required for the successful production of satisfactory tablets. Shelf or tray drier and fluidized-bed drier can be used for this purpose.

The dried granules are passed through a screen of smaller size than that used to prepare the moist granules. The size of the final granules is dependent on the size of the punches (and hence the final tablet size). Screens of 14- to 20-mesh size are generally used for this purpose. After dry screening, the dried and screened granules can be separated into coarse and fine granules by shaking them on a 250-mesh sieve. An appropriate quantity of lubricant is passed through a 200-mesh sieve. This is mixed with the fine granules before the coarse granules are incorporated. The quantity of lubricant used varies from one formulation scientist to another but usually ranges from about 0.1% to 5% of the weight of the granulation.

Suitable lubricants for use in wet granulation include, but are not limited to magnesium stearate, calcium stearate, stearic acid, wax, hydrogenated vegetable oil, talc, and starch.

A suitable dry granulation process includes weighing formulation ingredients in appropriate quantities. The excipients and active ingredient(s) should be in finely divided form, otherwise, particle size reduction should be carried out.

The weighed formulation ingredients are mixed in a powder mixer until a uniform powder mix is achieved. In embodiments, half the lubricant in the formula can be added at this stage to enhance powder flow during slugging and to prevent sticking of compressed powder on the die during precompression.

The mixed ingredients can then be compressed into flat large tablets or pellets, referred to as precompression (a.k.a., slugging) and the compacts made in the process typically have a sides of about 25 mm diameter by about 10-15 mm thick. Compression of mixed powders into slugs can be achieved by slugging technique or roller compaction. The pressure used to produce the slugs is usually less than that used in the final compression.

Following slugging, the slugs are broken into smaller pieces using a hammer mill or other conventional milling equipment. The milled slugs are screened to produce uniform granules. After screening, the remaining lubricant and other extragranular excipients such as disintegrant, glidant etc. as described herein, can be added to the granulation and mixed gently to achieve a uniform blend. The mixed granules can be compressed into tablets using either a single or rotary tablet press fitted with appropriate punches and dies. Similar to wet granulation, tablets manufactured by dry granulation method may be coated if the need arises. The disintegrant can be added to the weighed formulation at the beginning of the process (intragraular) or to the screened milled slugs (extragranular) and sometimes in both steps (intragranular extragranular). The formed granules can then be compressed into tablets or micro-tablets.

In certain embodiments, matrices can be formed using a melt-granulation or melt-extrusion technique. Generally, melt-granulation techniques involve melting a normally solid binder material, e.g. a wax, and incorporating a powder therein. Other ingredients can be added, for example, release modifying agents, diluents, lubricants, binders, granulating aids, colorants, flavorants and glidants that are conventional in the pharmaceutical art in amounts up to about 50% by weight of the particulate if desired. The quantities of these additional materials will be sufficient to provide the desired effect to the desired formulation.

According to embodiments, methods of preparing dosage forms as described herein can include a number of pharmaceutically acceptable preparation techniques (e.g., granulation, extrusion, compression, coating, encapsulation, etc. as discussed above) known to those of ordinary skill in the art. In embodiments, methods of preparation include granulating one or more components (e.g., at least one stress buster and at least one calming reliever) to form a matrix. Compressing (and optionally shaping) the matrix to form one or more micro-tablets using, for example, a tablet press. For example, the matrix can be compressed into tablets in a single rotary tablet press with a plurality of punch pairs. The punches can have a diameter of about 1 mm to about 10 mm and a hard chromium coated surface and can form cylindrical micro-tablets as shown in FIGS. 2A and 3A. In embodiments, the micro-tablets are coated with a coating comprising one or more aromatic oil (e.g., a mixture of mint oil and citrus oil, a mixture of mint oil and lavender oil, etc.). In embodiments, the micro-tablets may then be encapsulated within a pharmaceutically acceptable capsule (e.g., a soft gelatin capsule) known to those of ordinary skill in the art.

### Methods of Use

Further described herein are methods of using dosage forms and kits according to embodiments herein. In embodiments, disclosed are methods of supporting a human body's response to stress, that include administering to a human a dosage form, which may be a part of a kit, according to embodiments herein. In embodiments, the dosage form can be administered by the human as needed when the human anticipates stress, anxiety or both. In embodiments, the dosage form can be administered by the human in the afternoon or evening. According to various embodiments, the human can be administered any safe and suitable amount known to those of ordinary skill in the art. In embodiments, the human can be administered up to 1 serving, up to 2 servings, up to 3 servings, up to 4 servings, up to 5 servings, up to 6 servings, up to 7 servings, up to 8 servings, up to 9 servings, or up to 10 servings daily of the dosage form where each serving is, for example, 1 or 2 capsules, 1 or 2 tablets, 1 or 2 shots, etc.

According to embodiments, further disclosed are methods of supporting a human's response to anxiety disorder, comprising: administering to a human a dosage form according to embodiments described herein. The dosage form can be administered by the human as needed when the human anticipates stress, anxiety or both. In embodiments, the dosage form is administered by the human in the afternoon or evening. According to various embodiments, the human can be administered any safe and suitable amount known to those of ordinary skill in the art. In embodiments, the human can be administered up to 1 serving, up to 2 servings, up to 3 servings, up to 4 servings, up to 5 servings, up to 6 servings, up to 7 servings, up to 8 servings, up to 9 servings, or up to 10 servings daily of the dosage form where each serving is, for example, 1 capsule, 1 tablet, 1 shot, etc.

According to further embodiments, disclosed herein are methods of supporting a human's response to stress, comprising inducing a sensory stimulation invoking a sensory nervous system response, the sensory stimulation causing an acute reduction in stress in a human. In embodiments, the method includes delivering ingredients to target key receptors or other targets within the central nervous system, the ingredients indirectly driving an autonomic nervous system response causing a longer term reduction in stress of the human, wherein the inducing sensory stimulation and delivering ingredients causes a dual stimulation. In further embodiments, method includes providing, based on the dual stimulation induced by the sensory stimulation and delivered ingredients, a synergistic relaxation response modulated by the autonomic nervous system. In embodiments, the sensory stimulation provides a sensory distraction.

According to embodiments, disclosed herein are methods of supporting a human's response to anxiety, comprising inducing a sensory stimulation invoking a sensory nervous system response, the sensory stimulation causing an acute reduction in anxiety in a human. In embodiments, the method includes delivering ingredients to target key receptors or other targets within the central nervous system, the ingredients indirectly driving an autonomic nervous system response causing a longer term reduction in anxiety of the human, wherein the inducing sensory stimulation and delivering ingredients causes a dual stimulation. In embodiments the method includes providing, based on the dual stimulation induced by the sensory stimulation and delivered ingredients, a synergistic relaxation response modulated by the autonomic nervous system.

According to embodiments, disclosed herein is a method of treating anxiety disorder or depression, comprising: administering to a patient in need thereof a dosage form according to embodiments herein. In embodiments, the dosage form can include at least one additional active agent, for example, an anti-anxiety drug or an anti-depressant drug.

In yet further embodiments, disclosed herein is a method of using a kit to combine sensory disruption together with administering at least one dosage form to achieve a synergistic effect that assists the body's response to stress and to achieve allostasis. In embodiments, the method includes opening and holding a container housing the dosage forms under a person's nose. Exhaling then breathing in through the nose to draw breath into the bell. Optionally, the method includes pausing for about 1 second to about 10 seconds, and then optionally exhaling from the chest while imagining the belly button touching the spine. In embodiments, the method can include repeating the breathing and exhaling as desired and/or optionally for a particular number of times, for example, 5, 6, 7, 8, 9, 10, 15 or 20 times. In embodiments, the method includes orally administering the dosage form and tasting the dosage form while swallowing.

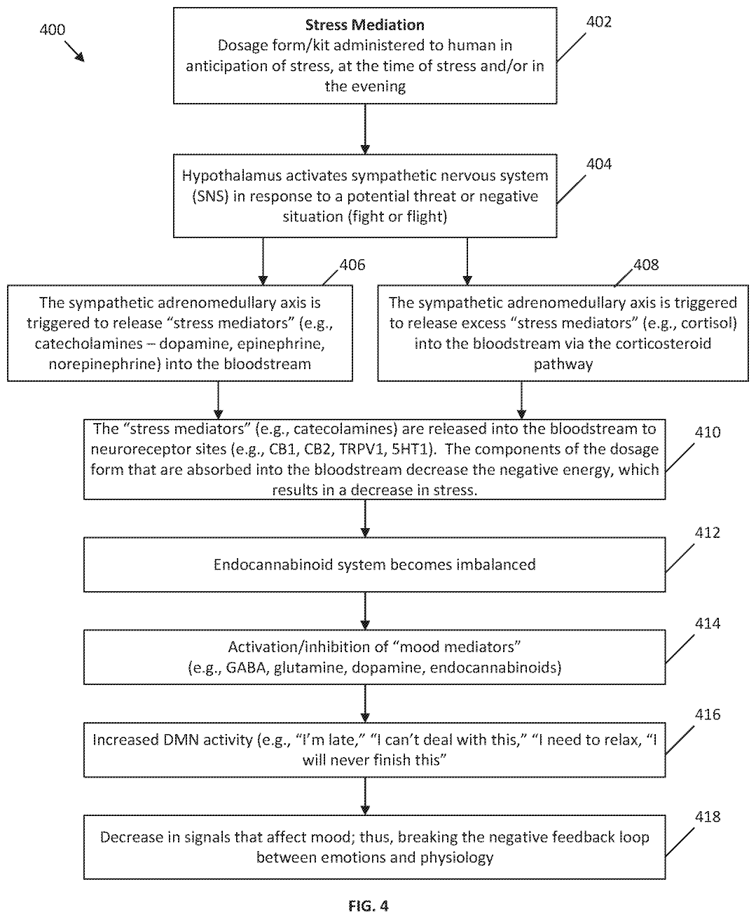

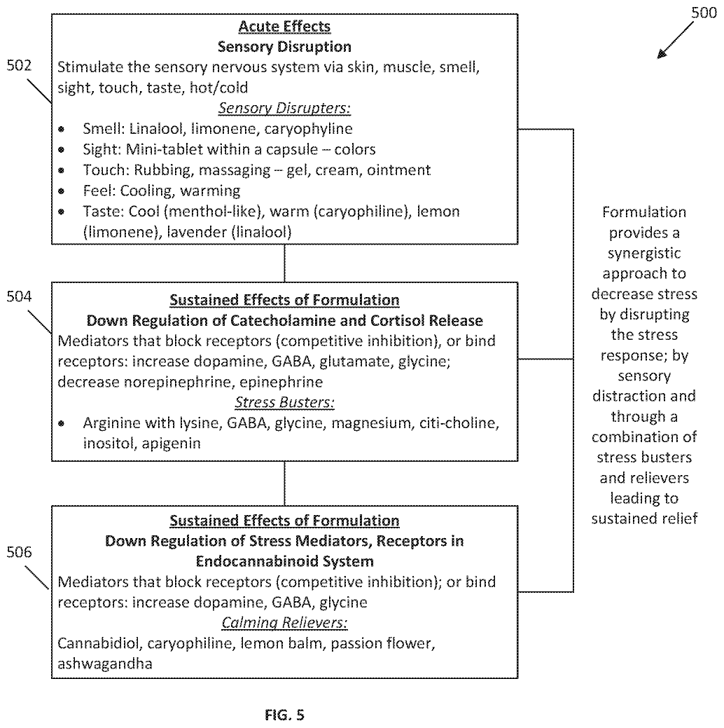

FIG. 4 is a flowchart showing the mediation of stress in the body 400 upon administration of a dosage form and/or kit according to embodiments herein. At block 402 a dosage form and/or kit is administered to a human in anticipation of stress, at the end of the day while experiencing stress and/or at the time of stress. At block 406 the sympathetic adrenomedullary axis is triggered to release “stress mediators” including catecholamines (e.g., dopamine, epinephrine, norepinephrine) into the bloodstream. At block 408 the sympathetic adrenomedullary axis also is triggered to release cortisol into the bloodstream via the corticosteroid pathway.

At block 410 the “stress mediators” (e.g., the catecholamines) are released into the bloodstream to neuroreceptor sites (e.g., CB1, CB2, TRPV1, 5HT1). The components of the dosage form that are absorbed into the blood stream decrease the negative energy, which results in a decrease in stress.

At block 412 the endocannabinoid system becomes imbalanced. At block 414, “mood mediators” (e.g., GABA, glutamine, dopamine, endocannabinoids) are activated/inhibited to help rebalance the body's systems. At block 416 there is increased DMN activity (e.g., “I'm late,” “I can't deal with this,” “I need to relax,” “I will never finish this”). At block 418 there is a decrease in signals that affect mood; thus, breaking the negative feedback loop between emotions and physiology

At block 404, upon experiencing stress, the hypothalamus activates the SNS in response to a potential threat or negative situation (“fight or flight”).

FIG. 5 is a flowchart showing the synergistic combination of effects 5from dosage forms and kits according to embodiments herein. At block 5 the acute effects of the sensory disruption that stimulates the sensory nervous system via skin, muscle, smell, sight, touch, taste, hot/cold, etc. Sensory disrupters targeting smell include, but are not limited to, aromatic oils such as linalool, limonene and caryophyllene. Sensory disrupters targeting sight including, but are not limited to the look of the dosage form (e.g., mini-tablet within a capsule, colors, etc.). Sensory disrupters targeting touch including, but are not limited to rubbing, massaging—gel, cream, ointment—feel of the container, etc. Sensory disrupters targeting feel include, but are not limited to components that provide cooling and warming sensations. Sensory disrupters targeting taste include, but are not limited to cool (menthol-like), warm (caryophyllene), lemon (limonene), lavender (linalool) and combinations thereof.

At block 504, the sustained effects of dosage forms according to embodiments herein include the down regulation of catecholamines and cortisol release. Mediators that block receptors (competitive inhibition), or bind receptors can increase dopamine, GABA, glutamate, glycine and/or decrease norepinephrine, epinephrine. Stress busters suitable to provide sustained effects include, but are not limited to arginine, lysine, GABA, glycine, magnesium, citi-choline, inositol, apigenin, valerian and combinations thereof.

At block 506, the sustained effects of dosage forms according to embodiments herein include the down regulation of stress mediators and receptors in the endocannabinoid system. Mediators that block receptors (competitive inhibition) or bind receptors can increase dopamine, GABA and glycine. Calming relievers suitable to provide sustained effects include, but are not limited to cannabinoids (e.g., cannabidiol), caryophyllene, lemon balm, passion flower, ashwagandha and combinations thereof.

Formulations according to embodiments herein provide a synergistic approach to decrease stress by disrupting the stress response. By sensory distraction and through a combination of sensory disrupters, stress busters and calming relievers, the body can achieve sustained relief and ultimately allostasis.

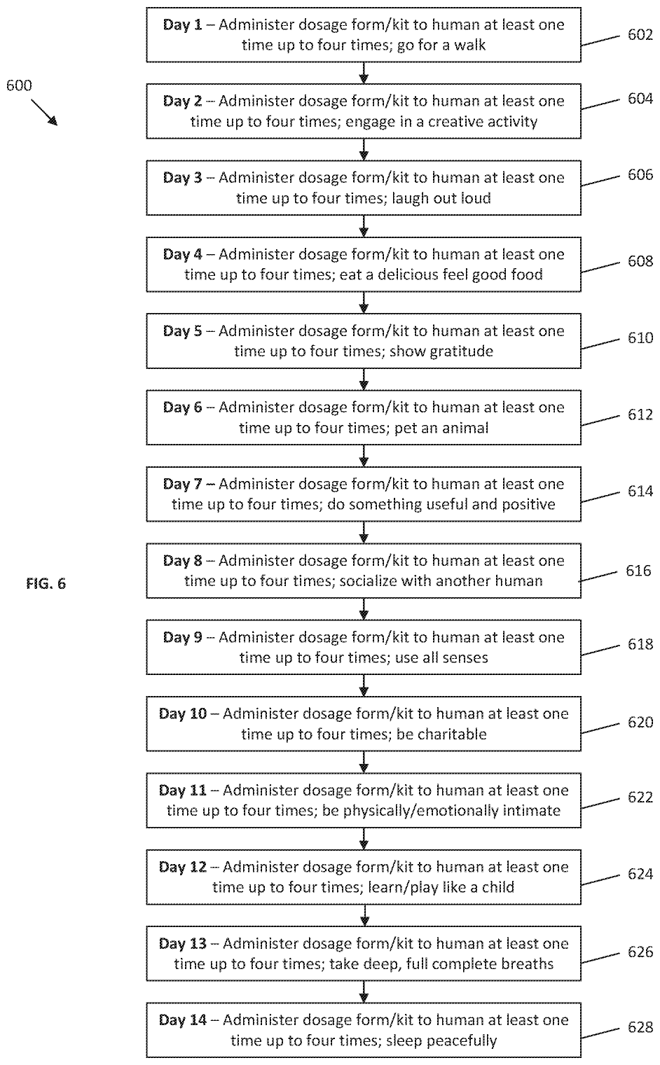

FIG. 6 is a flowchart showing an example 14-day administration cycle of dosage forms and kits according to embodiments herein. It should be noted that any order of the sensor disrupters identified in FIG. 6 can be used during the 14-day period. It should also be noted that the method can be practiced using any suitable sensory disrupters over any suitable number of days, e.g., 1 Day to 90 days, or 2 Days to 60 Days, or 5 Days to 45 Days, or 7 Days to 30 Days, or 7 Days, or 14 Days, or 28 Days and so on. Referring to FIG. 6, at block 602 on Day 1, administration of the dosage form is coupled with the sensory disrupter of going for a walk. As shown in block 602, the dosage form can be administered up to 4 times daily although, as discussed above, any suitable and safe number of servings (e.g., up to 10 servings daily) can be administered to the human on any of the Days. At block 604 on Day 2, administration of the dosage form is coupled with engaging in a creative activity. At block 606 on Day 3, administration of the dosage form is coupled with laughing out loud. At block 608 on Day 4, administration of the dosage form is coupled with eating a delicious feel good meal. At block 610 on Day 5, the dosage form is administered together with showing gratitude. At block 612 on Day 6, the dosage form is administered together with petting an animal. At block 614 on Day 7, administration of the dosage form is coupled with doing something useful and positive. At block 616 on Day 8, administration of the dosage form is coupled with socializing with another human being. At block 618 on Day 9, administration of the dosage form is coupled with the sensory disruption of using all senses. At block 620 on Day 10, administration of the dosage form is coupled with being charitable. At block 622 on Day 11, administration of the dosage form is coupled with being physically and/or emotionally intimate with another. At block 624 on Day 12, administration of the dosage form is coupled with learning and/or playing like a child. At block 626 on Day 13, administration of the dosage form is coupled with taking deep, full, complete breaths. At block 628 on Day 14, administration of the dosage form is coupled with sleeping peacefully.

In embodiments, methods of use can include administering dosage forms and kits according to embodiments herein daily for a period of 1 Day to 90 days, or 2 Days to 60 Days, or 5 Days to 45 Days, or 7 Days to 30 Days, or 7 Days, or 14 Days, or 28 Days and so on.

### EXAMPLES

### Example 1

### Formulation for Daytime

A daytime formulation was prepared as a dietary supplement to help the body's stress response. The ingredients shown in Table 1 were granulated and combined to form a matrix. One portion of the matrix was colored with beet extract to provide an orange color as a sensory disrupter. The matrices were compressed in a tablet press to form white and orange micro-tablets having a diameter of about 1 mm to about 10 mm, or about 5 mm. The micro-tablets were coated with a coating including a mixture of mint oil and citrus oil. In embodiments, the micro-tablets were encapsulated within a pharmaceutically acceptable soft shell capsule.

The resulting dosage form is shown in FIG. 3A. The dosage form was formulated to provide a controlled release of the components within the micro-tablets. The capsule shell also included a mint oil and a citrus oil as sensory disrupters in immediate release form. In the kit (FIG. 3B), the container included a matching orange color and upon opening the container lid, a citrus mint aroma diffused from the container. The daytime formulation was formulated to be taken during the day in anticipation of stress. The dosage was one capsule up to four times daily. Upon placing the dosage form in the mouth and swallowing, the dosage form tasted like a citrus mint flavor.

TABLE 1
Ingredients for Daytime Formulation
Ingredients in Daytime Formulation

| Ingredient | Description | Effects | Notes |
|---|---|---|---|
| Vegan Vitamin D3 | Vitamin D3 is | Shown to support | While the amount of |
| Oil | a fat-soluble | antioxidant and anti- | vitamin D3 included in |
| About 5 | nutrient. It is | inflammatory activity, | the Daytime product |
| mcg/capsule to | one of 24 | which is known to | may be less than the |
| about 100 | micronutrients | preserve the function | RDA and serving sizes |
| mcg/capsule | critical for | of immune cells | used in clinical trials, |
| | human | against homeostatic | the combinatory effect |
| | survival. | disturbances caused | of vitamin D3 and |
| | | by oxidative stress | other ingredients may |
| | | and inflammation. | support immune |
| | | | function when taken |
| | | | with a healthy diet and |
| | | | exercise. |
| Ahi flower Oil | Derived from | Omega-3 | Omega-3 oils are |
| About 100 | Buglossoides | polyunsaturated fatty | useful to modulate |
| mg/capsule to | arvensis | acids (PUFAs) are | inflammation and |
| about 500 | seeds, it is a | known to play critical | support a strong |
| mg/capsule | vegan omega-3 | roles in neuronal cell | immune system. |
| | oil that is | function as well as | |
| | rich in | inflammatory and | |
| | eicosapentaenoic | immune reactions | |
| | acid (EPA) and | involved in | |
| | docosahexaenoic | neuropsychiatric | |
| | acid (DHA). | disease states. | |
| MCT Oil | A medium | MCT oil can assist | MCT oils are useful to |
| About 50 | chain | with fat and nutrient | modulate |
| mg/capsule to | triglyceride | absorption issues, | inflammation and |
| about 275 | made from | weight loss, appetite | support a strong |
| mg/capsule | coconut or | control, extra energy | immune system. |
| | palm kernel | for exercise and | |
| | oil. | inflammation. | |
| L-Theanine | L-Theanine is | L-Theanine has | Clinical evidence |
| About 25 | one of the | various effects on the | suggests that even low |
| mg/capsule to | predominant | brain, such as | serving sizes of L- |
| about 250 | amino acids | neuroprotective | Theanine may boost |
| mg/capsule | ordinarily | activity, mood- | mental calmness in |
| | found in green | enhancement, and | healthy adults |
| | tea and is | relaxation. Studies | experiencing stress, |
| | useful as a | suggest it impacts the | which is supportive of |
| | relaxing | formation of the | the amounts used in |
| | agent. | inhibitory | the daytime |
| | | neurotransmitter | formulation. |
| | | GABA in the central | |
| | | nervous system. | |
| | | GABA blocks | |
| | | excessive stimulation | |
| | | of neuronal cells and | |
| | | may, therefore, have a | |
| | | key role in producing | |
| | | a relaxation effect. | |
| | | Testing shows | |
| | | L-Theanine increases | |
| | | brain alpha wave | |
| | | activity; a well- | |
| | | recognized index for | |
| | | relaxation. | |
| 5-Hydroxy | 5-HTP can be | A naturally occurring | Low dietary intake of |
| tryptophan | extracted from | amino acid building | 5-HTP (60-150 mg) |
| (5-HTP) | the seeds of | block often used as a | is effective in |
| (Griffonia | Griffonia | supplement to | elevating mood. |
| simplicifolia) | simplicifolia, | enhance biosynthesis | |
| About 2.5 | which is a | of the | |
| mg/capsule to | tropical shrub | neurotransmitter | |
| about 10 | native to West | serotonin. In the | |
| mg/capsule | Africa. | brain, 5-HTP is made | |
| | | from the amino acid | |
| | | tryptophan. The body | |
| | | absorbs tryptophan, | |
| | | converts it to 5-HTP | |
| | | then forms it into | |
| | | serotonin. Serotonin | |
| | | is an important | |
| | | compound that is well | |
| | | known to regulate | |
| | | mood, appetite, stress | |
| | | levels, among several | |
| | | other important | |
| | | functions. | |
| Valerian Root | Valerian is | Its natural relaxing | Clinical evidence is |
| Officinalis | useful as a | properties are exerted | supportive of the |
| CO2 extract | sedative and | through the | amount of valerian |
| About 0.5 | anti-anxiety | modulation of the | included in the |
| mg/capsule to | treatment. | GABA system, | Daytime formulation |
| about 10 | | similar to that of L- | for supporting |
| mg/capsule | | Theanine. Valerian | relaxation, without |
| | | root has been shown | inducing sedation, in |
| | | to reach the brain | healthy individuals |
| | | within 15 minutes of | experiencing nervous |
| | | taking it. | unrest. |
| Relora ® | Relora ® is a | Anxiety-reducing | The suggested serving |
| About 25 | blend of bark | properties in animal | of 1-2 capsules, daily, |
| mg/capsule to | extracts from | models. Bark of M. | of the Daytime |
| about 250 | Phellodendron | officinalis is useful | formulation may be |
| mg/capsule | amurense and | for antioxidant, | effective in reducing |
| | Magnolia | detoxifying and | stress and supporting |
| | officinalis. | relaxing potential. | relaxation in healthy |
| | | | individuals with |
| | | | anxiety, when taken |
| | | | along with a healthy |
| | | | diet and exercise. |
| PharmaGABA ™ | PharmaGABA ™ | Similar to the | Overall, the amount of |
| About 25 | provides a | function of L- | GABA indicated in the |
| mg/capsule to | natural source | Theanine, GABA | Daytime product is |
| about 250 | of GABA | may also be an | comparable to the |
| mg/capsule | derived from a | important compound | serving sizes used in |
| | fermentation | that increases alpha | clinical trials, and |
| | process. | wave activity in the | thereby may be |
| | | brain, thereby induce | effective in reducing |
| | | relaxation | stress and promoting |
| | | | relaxation in healthy |
| | | | individuals. |
| Sensoril ® | Native to | Also classified as an | Clinical evidence is |
| (Ashwagandha) | India and | adaptogen, which is a | supportive of |
| About 25 | Middle East, | substance that helps | ashwagandha extracts |
| mg/capsule to | is useful in | the body withstand | for a variety of stress- |
| about 125 | Ayurvedic | environmental | related symptoms, |
| mg/capsule | medicine for | stressors and thereby, | even at the low |
| | its calming, | promote resilience to | amounts of 125 mg |
| | antidepressant, | stress. The | included in the |
| | sleep | phytochemicals in | Daytime product. |
| | promoting, | ashwagandha | |
| | effects. | counteract negative | |
| | | effects of stress by | |
| | | increasing levels of | |
| | | GABA signaling and | |
| | | preventing | |
| | | overstimulation of the | |
| | | brain, similar to the | |
| | | activity of L-Theanine. | |
| Beta | A | Through selective | Though the amount of |
| Caryophyllene | phytochemical | binding to CB1 and | Beta-Caryophyllene |
| About 0.5 | naturally | CB2 receptors, Beta- | indicated in the |
| mg/capsule to | found in many | Caryophyllene may | Daytime product is |
| about 10 | edible plants | exert anti- | slightly higher than |
| mg/capsule | such as basil, | inflammatory and | amounts in various |
| | lemon balm, | stress-balancing | studies, scientific |
| | clove, and | activity. | evidence is supportive |
| | hemp. | | of its safe |
| | | | consumption in |
| | | | healthy adults. |

### Example 2

### Formulation for Evening

An evening formulation was prepared as a dietary supplement to help the body's stress response. The ingredients shown in Table 2. were granulated and combined to form a matrix. One portion of the matrix was colored with spirulina extract to provide a lavender color as a sensory disrupter. The matrices were compressed in a tablet press to form white and purple micro-tablets having a diameter of about 1 mm to about 10 mm, or about 5 mm. The micro-tablets were coated with a coating including a mixture of mint oil and lavender oil. In embodiments, the micro-tablets were encapsulated within a pharmaceutically acceptable soft shell capsule.

The resulting dosage form is shown in FIG. 2A. The dosage form was formulated to provide a controlled release of the components within the micro-tablets. The capsule shell also included a mint oil and a lavender oil as sensory disrupters in immediate release form. In the kit (FIG. 2B), the container included a matching purple color and upon opening the container lid, a lavender mint aroma diffused from the container. The evening formulation was formulated to be taken at the end of the day to help the body recover, reset and rebalance after experiencing stress. The dosage was one capsule up to four times daily. Upon placing the dosage form in the mouth and swallowing, the dosage form tasted like a lavender mint flavor.

TABLE 2
Ingredients for Evening Formulation

| Ingredient | Description | Reported effects | Notes |
|---|---|---|---|
| Vegan Vitamin D3 | Vitamin D3 is | Shown to support | Although the amount |
| Oil | a fat-soluble | antioxidant and anti- | of vitamin D3 in the |
| About 5 | nutrient. It is | inflammatory | Evening formulation |
| mcg/capsule to | one of 24 | activity, which is | may be less than the |
| about 100 | micronutrients | known to preserve the | RDA and the serving |
| mcg/capsule | critical for | function of immune | sizes used in clinical |
| | human | cells against | trials, the combinatory |
| | survival. | homeostatic | effect of vitamin D3 |
| | | disturbances caused | and other ingredients |
| | | by oxidative stress | may support immune |
| | | and inflammation. | function when taken |
| | | | with a healthy diet and |
| | | | exercise. |
| Ahi flower | Derived from | These omega-3 | Although clinical trials |
| About 100 | Buglossoides | polyunsaturated fatty | using Ahi flower oil |
| mg/capsule to | arvensis | acids (PUFAs) are | are limited in scope, |
| about 500 | seeds, is a | play critical roles in | omega-3 oils have a |
| mg/capsule | vegan omega-3 | neuronal cell function | long-standing history |
| | oil rich in | and inflammatory and | in the regulatory arena |
| | eicosapentaenoic | immune reactions | for their potential to |
| | acid (EPA) and | involved in | modulate |
| | docosahexaenoic | neuropsychiatric | inflammation and |
| | acid (DHA). | disease states. | support a strong |
| | | | immune system. |
| MCT Oil | A medium | MCT oil can assist | MCT oils are useful to |
| About 50 | chain | with fat and nutrient | modulate |
| mg/capsule to | triglyceride | absorption issues, | inflammation and |
| about 275 | made from | weight loss, appetite | support a strong |
| mg/capsule | coconut or | control, extra energy | immune system. |
| | palm kernel | for exercise and | |
| | oil. | inflammation. | |
| Passionflower | Useful as a | There is scientific | Although the amount |
| (Passiflora | sedative and | evidence that | of passionflower |
| incarnata) | to treat | passionflower may | included in the |
| About 5 | anxiety. | decrease anxiety via | Evening formulation |
| mg/capsule to | | interaction with the | may be less than the |
| about 100 | | GABA(A) receptor in | serving sizes used in |
| mg/capsule | | laboratory | clinical trials, the |
| | | experiments, and | combinatory effect of |
| | | thereby promotes a | the herbal ingredients |
| | | calming effect. | may support the use of |
| | | Studies have | passionflower for |
| | | also shown | restorative sleep in |
| | | passionflower | healthy adults without |
| | | reduced inflammation | inducing sedation or |
| | | in brain cells. | impairment. |
| | | Because stress is well | |
| | | known to cause | |
| | | inflammation and | |
| | | disrupt the sleep | |
| | | cycle, the anti- | |
| | | inflammatory and | |
| | | sedative compounds | |
| | | abundant in | |
| | | passionflower may | |
| | | have diverse soothing | |
| | | properties. | |
| Lemon Balm | A lemon | Luteolin, a flavonoid | A small amount of |
| (Melissa | scented herb | of lemon balm, has | lemon balm in the |
| officinalis) | useful to | calming and anti- | Evening formulation is |
| About 5 | reduce | inflammatory effects | safe and may elicit |
| mg/capsule to | anxiety, stress, | when administered | calming and immune- |
| about 50 | and to aid in | both topically and | enhancing effects. |
| mg/capsule | sleep. | orally, although the | |
| | | exact mechanism of | |
| | | action is uncertain. | |
| | | Recent in vitro | |
| | | research suggests that | |
| | | low amounts of | |
| | | lemon balm treatment | |
| | | (e.g., 100 mg) may | |
| | | modulate the central | |
| | | nervous system | |
| | | through interacting | |
| | | with GABA receptors | |
| | | to enhance mood states. | |
| Chamomile Flower | Chamomile is | Abundant in apigenin, | When taken along |
| (Matricaria | ueful to | a compound related to | with a healthy diet and |
| chamomilla) | manage a | anti-inflammatory, | exercise, the |
| CO₂-extract | wide range of | antiseptic and anti- | synergistic effects of |
| (organic) | health | histaminic effect. | chamomile, |
| About 0.25 | conditions due | Inflammation is a | ashwagandha and |
| mg/capsule to | to its immune | complex biological | passionflower |
| about 5 | modulating | response that can lead | indicated in the |
| mg/capsule | and relaxant | to tissue damage and | Evening formulation |
| | properties. | a weakened immune | may have the potential |
| | | system if it occurs for | to balance stress levels |
| | | a prolonged period. | and support healthy |
| | | By reducing | immune function. |
| | | stimulation of | |
| | | inflammatory | |
| | | pathways, | |
| | | chamomile, may be | |
| | | essential in | |
| | | maintaining a normal | |
| | | and healthy immune | |
| | | system. | |
| Valerian Root | Useful as a | Its natural relaxing | Clinical evidence |
| Officinalis CO₂ | sedative and | properties are exerted | supports that the |
| extract | anti-anxiety | through the | amount of valerian |
| About 5 | treatment. | modulation of the | included in the |
| mg/capsule to | | GABA system, | Evening formulation is |
| about 10 | | similar to that of L- | sufficient to support |
| mg/capsule | | Theanine. Valerian | relaxation, without |
| | | root has been shown | inducing sedation, in |
| | | to reach the brain | healthy individuals |
| | | within 15 minutes of | experiencing nervous |
| | | taking it. | unrest. |
| Relora ® | Relora ® is a | Anxiety-reducing | The suggested serving |
| About 25 | blend of bark | properties in animal | of 1-2 capsules, daily, |
| mg/capsule to | extracts from | models. Bark of M. | of the Evening |
| about 250 | Phellodendron | officinalis is useful | supplement can be |
| mg/capsule | amurense and | for antioxidant, | effective in reducing |
| | Magnolia | detoxifying and | stress and supporting |
| | officinalis. | relaxing potential. | relaxation in healthy |
| | | | individuals with |
| | | | anxiety, when taken |
| | | | along with a healthy |
| | | | diet and exercise. |
| PharmaGABA | PharmaGABA ™ | Similar to the | Overall, the amount of |
| About 25 | provides a | function of L- | GABA indicated in the |
| mg/capsule to | natural source | Theanine, GABA | Evening formulation is |
| about 250 | of GABA | may also be an | comparable to the |
| mg/capsule | derived from a | important compound | serving sizes used in |
| | fermentation | that increases alpha | clinical trials, and |
| | process. | wave activity in the | thereby may be |
| | | brain, thereby induce | effective in reducing |
| | | relaxation. | stress and promoting |
| | | | relaxation in healthy |
| | | | individuals. |
| Sensoril ® | Native to | Also classified as an | Clinical evidence |
| (Ashwagandha) | India and | adaptogen, which is a | supports the use of |
| About 25 | Middle East, | substance that helps | ashwagandha extracts |
| mg/capsule to | useful in | the body withstand | for a variety of stress- |
| about 125 | Ayurvedic | environmental | related symptoms, |
| mg/capsule | medicine for | stressors and thereby, | even at the low |
| | its calming, | promote resilience to | amounts of 125 mg |
| | antidepressant, | stress. The | included in the |
| | sleep | phytochemicals in | Evening product. |
| | promoting, | ashwagandha | |
| | effects. | counteract the | |
| | | negative effects of | |
| | | stress by increasing | |
| | | levels of GABA | |
| | | signaling and | |
| | | preventing | |
| | | overstimulation of the | |
| | | brain, similar to the | |
| | | activity of L-Theanine. | |
| Beta Caryophyllene | A | Through selective | Though the amount of |
| About 5 | phytochemical | binding to CB1 and | Beta-Caryophyllene |
| mg/capsule to | naturally | CB2 receptors, Beta- | indicated in the |
| about 10 | found in many | Caryophyllene may | Evening product is |
| mg/capsule | edible plants | exert anti- | slightly higher than the |
| | such as basil, | inflammatory and | amounts in the |
| | lemon balm, | stress-balancing | aforementioned |
| | clove, and | activity. | studies, scientific |
| | hemp. | | evidence is supportive |
| | | | of its safe |
| | | | consumption in |
| | | | healthy adults. |

### Example 3

### Daytime Formulation with At Least One Additional Active (Prophetic)

An oral dosage form is prepared as a tablet including the ingredients from Example 1 compressed together with the anti-anxiety drug buspirone. The ingredients and buspirone are formed into granules to provide a matrix, where the matrix is compressed into a tablet. The tablet includes a beet colorant to provide an orange color. The tablet is coated with a mixture of citrus oil and mint oil such that when a container holding several of the tablets is opened, a citrus mint aroma diffuses out, which provides a sensory distraction. Upon administering a single tablet (dosage: 1 tablet up to 4× daily), the dosage form tastes of citrus and mint.

### Example 4

### Evening Formulation with At Least One Additional Active (Prophetic)

An oral dosage form is prepared with the ingredients from Table 2 mixed together with the drug dronabinol (synthetic THC). The ingredients from Table 2 are formed into granules and combined to provide a matrix. Dronabinol in powder form is combined with the matrix and the resulting matrix is compressed to form micro-tablets. A portion of the matrix is combined with a spirulina extract to provide a lavender color. The resulting matrices are compressed with a tablet press to form white and purple micro-tablets having a size of about 1 mm to about 10 mm, or about 5 mm. A soft gelatin capsule shell is prepared using a mixture of lavender oil and mint oil and the micro-tablets are encapsulated in the soft gelatin capsule. When a container holding several of the dosage forms is opened, a lavender mint aroma diffuses out, which provides a sensory distraction. Upon administering a single capsule (dosage: 1 capsule up to 4× daily), the dosage form tastes of lavender and mint.

### Example 5

### Evening Formulation with At Least One Additional Active (Prophetic)

An oral dosage form is prepared with the ingredients from Table 2 and the drug dronabinol. The ingredients from Table 2 are granulated and combined to provide a matrix. A portion of the matrix is combined with a spirulina extract to provide a lavender color. The matrices are compressed using a tablet press to form white and purple micro-tablets having a size of about 1 mm to about 10 mm, or about 5 mm. The micro-tablets are coated with a coating including a mixture of lavender oil and mint oil. The micro-tablets are encapsulated in one part of a soft shell capsule while the dronabinol in liquid form is encapsulated in another part of the soft shell capsule. The soft shell capsule is also coated with a coating comprising lavender oil and mint oil. When a container holding several of the dosage forms is opened, a lavender mint aroma diffuses out, which provides a sensory distraction. Upon administering a single capsule (dosage: 1 capsule up to 4x daily), the dosage form tastes of lavender and mint.

### Example 6

### Daytime Formulation with At Least One Additional Active (Prophetic)

An oral dosage form is prepared as a tablet including the ingredients from Table 1 compressed together with cannabidiol (CBD). The ingredients from Table 1 are granulated and combined to form a matrix. CBD in powder form is combined with the matrix. A portion of the resulting matrix is combined with beet extract to provide an orange color. The resulting matrices are compressed to form white and orange tablets. The tablets are coated with a mixture of citrus oil and mint oil. When a container holding several of the dosage forms is opened, a citrus mint aroma diffuses out, which provides a sensory distraction. Upon administering a single tablet (dosage: 1 capsule up to 4x daily), the dosage form tastes of citrus and mint.

### Example 7

### Evening Formulation with At Least One Additional Active (Prophetic)

The formulation of Example 4 is prepared in the same way except that the dronabinol in powdered form is replaced with a mixture of dronabinol and CBD in powdered form. The resulting dosage form provides a synergistic effect from the combination of the dronabinol and the CBD in addition to the effects from the stress disrupters, stress busters and calming relievers.

### Example 8

### Topical Formulation (Prophetic)

A topical formulation is prepared that produces a warming sensation or a cooling sensation upon application. The ingredients from Table 1 are granulated and combined to form a matrix. The matrix is combined with a lotion. Essential oils, for example, lavender oil and mint oil, and a terpene, for example, linalool, are combined in the lotion mixture. The essential oils produce a pleasant calming aroma or scent. In the warming formulation, the lotion is combined with, for example, cinnamon essential oil. In the cooling formulation, the lotion is combined with, for example, menthol.

### Example 9

### Topical Patch Formulation (Prophetic)

A topical patch is prepared according to any suitable method known to those of ordinary skill in the art. The topical patch contains the ingredients from Table 1 or Table 2. The ingredients are further combined with cinnamon essential oil for a warming patch or menthol for a cooling patch. The topical patch initiates either/or a warming or a cooling sensation upon application. The topical patch includes additional ingredients that optimize penetration into the skin resulting in mid-long term relief.

### Example 7

### Oral Formulation (Prophetic)

An oral formulation includes a clear capsule containing one or more stress disrupter, stress buster and calming reliever where an oil (such as hemp seed oil) is contained in a larger outer capsule, and one or more smaller clear capsule(s) contains the one or more stress disrupter, stress buster and calming reliever in a dried or powder form. The capsule provides a rapid release of ingredient(s) contained in the outer capsule (e.g., the hemp seed oil), and a sustained release of the ingredients in the smaller capsule(s).

### Example 8

### Oral Formulation (Prophetic)

An oral dosage form can include a formulation prepared using spray-drying or similar technology to form granules for a sachet packet as the dosage form. The finished dosage form is ingested directly via the oral cavity. The sachet could incorporate various ingredients that would invoke one or more of the following sensations: a cooling or heating sensation, bitter, sour or sweet taste upon ingestion via mouth. Another option would employ ingredients to invoke a mild “popping” tingling sensation (similar to “Pop Rocks”).

The use of headings and sections in the application is not meant to limit the disclosure; each section can apply to any aspect, embodiment, or feature of the disclosure. Only those claims which use the words “means for” are intended to be interpreted under 35 USC 112, sixth paragraph. Absent a recital of “means for” in the claims, such claims should not be construed under 35 USC 112. Limitations from the specification are not intended to be read into any claims, unless such limitations are expressly included in the claims.

When values or ranges of values are given, each value and the end points of a given range and the values there between may be increased or decreased by 20%, while still staying within the teachings of the disclosure, unless some different range is specifically mentioned.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the disclosure as if each value were specifically enumerated herein. In addition, smaller ranges between and including the upper and lower limits of a given range are contemplated and encompassed within the disclosure. The listing of exemplary values or ranges is not a disclaimer of other values or ranges between and including the upper and lower limits of a given range.

It is to be understood that the figures and descriptions of the disclosure have been simplified to illustrate elements that are relevant for a clear understanding of the disclosure, while eliminating, for purposes of clarity, other elements. Those of ordinary skill in the art will recognize, however, that these and other elements may be desirable. However, because such elements are well known in the art, and because they do not facilitate a better understanding of the disclosure, a discussion of such elements is not provided herein. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

1. A dosage form, comprising:
at least one sensory disrupter, optionally wherein the sensory disrupter comprises at least one of a terpene, linalool, limonene and caryophyllene;
at least one stress-buster, optionally wherein the stress buster comprises at least one of arginine, lysine, glycine, gamma amino butyric acid (GABA), magnesium, citi-choline, inositol and apigenin; and
at least one calming reliever, optionally wherein the calming reliever comprises at least one of cannabidiol, lemon balm, passionflower and ashwagandha.

2. The dosage form of claim 1, comprising a plurality of units, the plurality of units comprising at least one of the sensory disrupter, the stress-buster; and the calming reliever,
optionally wherein the plurality of units comprise a first plurality of units and a second plurality of units, and
optionally, wherein the plurality of units provide a controlled release of components within the plurality of units.

3. A dosage form, comprising:
a nervous system component, optionally wherein the nervous system component comprises at least one of gamma amino butyric acid, valerian root, 5-hydroxytryptophan, L-theanine, passionflower and lemon balm;
an immune system component, optionally wherein the immune system component comprises at least one of Ahi flower oil, Vitamin D3 and an aromatic oil; and
an endocrine system component, optionally wherein the endocrine system component comprises at least one of magnolia extract, phaelladendron extract and ashwagandha.

4. The dosage form of claim 3, comprising a plurality of units, the plurality of units comprising at least one of the nervous system component, the immune system component and the endocrine system component,
optionally wherein the plurality of units comprise a first plurality of units and a second plurality of units, and
optionally, wherein the plurality of units provide a controlled release of components within the plurality of units.

5. The dosage form of claim 2, wherein the plurality of units comprise at least one of particles, granules, beads, microspheres, micro-tablets and extrudates.

6. The dosage form of claim 2, wherein the first plurality of units is a different color from the second plurality of units,
optionally, wherein the color is derived from at least one of beets and spirulina.

7. The dosage form of claim 2, wherein the plurality of units are contained in a pharmaceutically acceptable capsule,
optionally, wherein the capsule comprises a mixture of natural oils and components, and
optionally, wherein the capsule provides an immediate release of the natural oils and components.

8. The dosage form of claim 7, wherein the capsule is clear or translucent.

9. The dosage form of claim 7, further comprising a coating on the capsule.

10. The dosage form of claim 1, comprising an aromatic oil.

11. The dosage form of claim 10, wherein the aromatic oil comprises at least one of a plant terpene, lavender oil, citrus oil, chamomile oil, rose oil, hyssop oil, mint oil, peppermint oil, spearmint oil, wintergreen oil, clary sage oil, rosemary oil, ylang ylang oil, myrrh oil, vetiver oil, lemongrass oil, orange oil, grapefruit oil, lemon oil, frankincense oil, helichrysum oil, cedarwood oil, basil oil, melaleuca oil, arborvitae oil, patchouli, clove oil, cassia oil, oregano oil, rosemary oil, thyme oil, mango oil, juniper oil, cinnamon oil, pepper oil, lilac oil, nutmeg oil, cumin oil, pine oil, parsley oil, orchid oil and cannabis oil,
optionally, wherein the plant terpene comprises at least one of myrcene, pinene, caryophyllene, limonene and terpinolene.

12. The dosage form claim 1,
wherein the dosage form provides an immediate release of at least one ingredient, or
wherein the dosage form provides a controlled release of at least one ingredient, or
wherein the dosage form provides an immediate release of at least one ingredient and a controlled release of at least one ingredient.

13. The dosage form of claim 1, wherein the dosage form is free of at least one of melatonin, diphenhydramine, caffeine, cannabinoids, cypress oil, eucalyptus oil, tea tree oil, rose oil and sandalwood oil.

14. The dosage form of claim 1, wherein the dosage form comprises a route of administration selected from the group consisting of oral, parenteral, subcutaneous, intravenous, intramuscular, intraperitoneal, transdermal, transmucosal, transurethral, sublingual, buccal, gingival, rectal, subcutaneous, transpulmonary, topical, nasal, inhalation and combinations thereof.

15. The dosage form of claim 1, further comprising at least one additional active ingredient,
optionally, wherein the at least one additional active ingredient comprises at least one of an anti-anxiety drug, an antidepressant, a cannabinoid, dronabinol, a prodrug thereof, a pharmaceutically acceptable salt thereof and combinations thereof,
optionally, wherein the anti-anxiety drug comprises at least one of a central nervous system depressant, a benzodiazepine, buspirone, buproprion, a prodrug thereof, a pharmaceutically acceptable salt thereof and combinations thereof,
optionally, wherein the benzodiazepine comprises at least one of alprazolam, chlordiazepoxide, clonazepam, diazepam, lorazepam, a prodrug thereof, a pharmaceutically acceptable salt thereof and combinations thereof,
optionally, wherein the central nervous system depressant comprises a barbiturate, a prodrug thereof, a pharmaceutically acceptable salt thereof and combinations thereof,
optionally, wherein the barbiturate comprises at least one of secobarbital, butabarbital, mephobarbital, pentobarbital, phenobarbital, amobarbital, a prodrug thereof, a pharmaceutically acceptable salt thereof and combinations thereof,
optionally, wherein the antidepressant comprises at least one of a selective serotonin reuptake inhibitor (S SRI), a selective noradrenaline reuptake inhibitor (SNRI), a tricyclic, a monoamine oxidase inhibitor (MAOI), a beta-blocker, a sedative, a prodrug thereof, a pharmaceutically acceptable salt thereof and combinations thereof,
optionally, wherein the SSRI comprises at least one of citalopram, escitalopram, fluvoxamine, cerixamine, fenfluramine, paroxetine, dapoxetine, dapoxetine, fluoxetine, ifoxetine, vortioxetine, sertraline, vilazodone, cyanodothiepin, nefazodone, a prodrug thereof and a pharmaceutically acceptable salt thereof,
optionally, wherein the SNRI comprises at least one of venlafaxine, desvenlafaxine, milnacipran, duloxetine, levomilnacipran, reboxetine, desipramine, maprotiline, lofepramine, mirtazapine, oxaprotilin, fezolamine, atomoxetine, bupropion, mianserin, duloxetine, a prodrug thereof and a pharmaceutically acceptable salt thereof,
optionally, wherein the tricyclic comprises at least one of clomiprmamine, imipramine, a prodrug thereof, a pharmaceutically acceptable salt thereof and combinations thereof,
optionally, wherein the MAOI comprises at least one of socarboxazid, phenelzine, selegiline, tranylcypromine, a prodrug thereof, a pharmaceutically acceptable salt thereof and combinations thereof, and
optionally, wherein the beta-blocker comprises at least one of propranolol, atenolol, a prodrug thereof, a pharmaceutically acceptable salt thereof and combinations thereof.

16. The dosage form of claim 1, wherein the dosage form is free of at least one of melatonin, diphenhydramine, caffeine, a cannabinoid, cypress oil, eucalyptus oil, tea tree oil, rose oil and sandalwood oil.

17. A dosage form, comprising:
vitamin D3;
ahi flower;
L. Theanine;
5 -hydroxytryptophan;
valerian root;
a mixture of phellodendron amurense and magnolia officinalis;
gama amino butyric acid;
ashwagandha;
beta caryophyllene;
citrus oil; and
mint oil,
optionally, wherein the dosage form is a pharmaceutically acceptable capsule comprising a combination of natural oils and ingredients,
optionally, wherein the capsule provides an immediate release of the natural oils and ingredients,
optionally, wherein the capsule is translucent or clear,
optionally, wherein the ingredients are in the form of a plurality of units,
optionally, wherein a first plurality of the plurality of units is a different color from a second plurality of the plurality of units,
optionally, wherein the plurality of units provide a controlled release of the ingredients, and
optionally, wherein the dosage form comprises at least one additional active ingredient.

18. A dosage form, comprising:
vitamin D3;
ahi flower;
passionflower;
lemon balm;
chamomile flower;
valerian root;
a mixture of Phellodendron amurense and Magnolia officinalis;
gama amino butyric acid;
ashwagandha;
beta caryophyllene;
lavender oil; and
mint oil,
optionally, wherein the dosage form is a pharmaceutically acceptable capsule comprising a combination of natural oils and ingredients,
optionally, wherein the capsule provides an immediate release of the natural oils and ingredients,
optionally, wherein the capsule is translucent or clear,
optionally, wherein the ingredients are in the form of a plurality of units,
optionally, wherein a first plurality of the plurality of units is a different color from a second plurality of the plurality of units,
optionally, wherein the plurality of units provide a controlled release of the ingredients, and
optionally, wherein the dosage form comprises at least one additional active ingredient.

19. A kit, comprising:
a container; and
at least one dosage form according to claim 1, the at least one dosage form removably positioned within the container,
optionally, wherein the kit further comprises an aromatic oil within the container,
optionally, wherein the kit further comprises external packaging, the container removably positioned within the external packaging,
optionally, wherein the kit further comprises an aromatic oil within the external packaging,
optionally, wherein at least one of the container and the external packaging comprises at least one of a textured material, a soft material, a wood material, a bamboo material and a calming color that, upon touch, sight or both, provides a sensory disruption to a human,
optionally wherein the aromatic oil within at least one of the container and the external packaging is within a canister, coated on an inner surface thereof, coated on an outer surface thereof, coated on an outer surface of the dosage form, contained within the dosage form, or a combination thereof.

20. The kit of claim 19, further comprising instructions for using the kit to reduce at least one of stress and anxiety,
optionally, wherein the instructions direct a human to:
open the container and hold under nose, exhale then breathe in through the nose drawing breath all the way into belly;
pause for about 1 second to about 10 seconds, then exhale from chest, imagine belly button touching spine, optionally, repeat breathing and exhaling; and
insert one dosage form into mouth, taste the dosage form while swallowing.

21. A method of supporting a human body's response to at least one of stress and anxiety, comprising: administering to a human a dosage form according to claims 1,
optionally, wherein the dosage form is administered by the human as needed when the human anticipates stress, anxiety or both,
optionally, wherein the dosage form is administered by the human in the afternoon or evening,
optionally, wherein the human is administered up to four servings daily of the dosage form.

22. A method of supporting a human's response to at least one of stress and anxiety, comprising:
inducing a sensory stimulation invoking a sensory nervous system response, the sensory stimulation causing an acute reduction in stress in a human;
administering a dosage form according to claim 1 to target key receptors or other targets within the central nervous system, the ingredients indirectly driving an autonomic nervous system response causing a longer term reduction in stress of the human, wherein the inducing sensory stimulation and delivering ingredients causes a dual stimulation; and
providing, based on the dual stimulation induced by the sensory stimulation and delivered ingredients, a synergistic relaxation response modulated by the autonomic nervous system,
optionally, wherein the sensory stimulation provides a sensory distraction.
